SEQUENCE LISTING (12 sequences)

SEQ ID NO: 1 - DNA, 20 bp, Artificial Sequence
  TLR2 Forward primer
gatgcctactgggtggagaa

SEQ ID NO: 2 - DNA, 20 bp, Artificial Sequence
  TLR2 Reverse primer
cgcagctctcagatttaccc

SEQ ID NO: 3 - DNA, 19 bp, Artificial Sequence
  TLR4 Forward primer
acagaagctggtggctgtg

SEQ ID NO: 4 - DNA, 21 bp, Artificial Sequence
  TLR4 Reverse primer
tctttaaatgcacctggttgg

SEQ ID NO: 5 - DNA, 20 bp, Artificial Sequence
  NOD1 Forward primer
ccacttcacagctggagaca

SEQ ID NO: 6 - DNA, 20 bp, Artificial Sequence
  NOD1 Reverse primer
tgagtggaagcagcattttg

SEQ ID NO: 7 - DNA, 20 bp, Artificial Sequence
  NOD2 Forward primer
gaatgttgggcacctcaagt

SEQ ID NO: 8 - DNA, 20 bp, Artificial Sequence
  NOD2 Reverse primer
caaggagcttagccatggag

SEQ ID NO: 9 - DNA, 20 bp, Artificial Sequence
  Rip2 Forward primer
ccattgagatttcgcatcct

SEQ ID NO: 10 - DNA, 20 bp, Artificial Sequence
  Rip2 Reverse primer
atgcgccactttgataaacc

SEQ ID NO: 11 - DNA, 20 bp, Artificial Sequence
  RPL13A Forward primer
catcgtggctaaacaggtac

SEQ ID NO: 12 - DNA, 20 bp, Artificial Sequence
  RPL13A Reverse primer
gcacgaccttgagggcagcc

(19) United States
(12) Patent Application Publication
(10) Pub. No.: US 2017/0007668 A1
(43) Pub. Date: Jan. 12, 2017

(54) Pharmaceutical Composition Comprising Stem Cells Treated with NOD2 Agonist or Culture Thereof for Prevention and Treatment of Immune Disorders and Inflammatory Diseases

(71) Applicant: KANG STEM BIOTECH CO., LTD, Seoul (KR)

(72) Inventors: Kyung Sun Kang, Seoul (KR); Hyung Sik Kim, Seoul (KR)

(21) Appl. No.: 15/231,223

(22) Filed: Aug. 8, 2016

Related U.S. Application Data
(63) Continuation-in-part of application No. 13/818,234, filed on Apr. 9, 2013, now Pat. No. 9,408,873.

(30) Foreign Application Priority Data
Aug. 23, 2010 (KR) 10-2010-0081640

(51) Int. Cl. A61K 38/17; A61K 9/00; C12N 5/0775; A61L 27/38; A61L 27/54; A61K 35/28; A61K 38/14

(52) U.S. Cl. CPC A61K 38/177; A61K 35/28; A61K 9/0019; A61K 38/14; A61L 27/3834; A61L 27/54; C12N 5/0665; A61L 2300/252; C12N 2501/02; C12N 2501/05; C12N 2501/052; C12N 2501/054

(57) ABSTRACT

The present invention relates to a pharmaceutical composition for the prevention or treatment of immune disorders and inflammatory diseases, comprising stem cells that are generated by culturing stem cells expressing Nucleotide-binding Oligomerization Domain protein 2 (NOD2) or a culture thereof. More particularly, the present invention relates to a method for suppressing immune responses or inflammatory responses of a subject, comprising the step of administering the pharmaceutical composition, the stem cells or culture thereof to the subject, a method for preparing an immunosuppressive drug or an anti-inflammatory drug using the stem cells or culture thereof, a graft comprising stem cells expressing NOD2, a method for preparing the graft, a composite comprising stem cells expressing NOD2, and a culture generated by culturing the NOD2-expressing stem cells.

### CROSS-REFERENCE TO RELATED APPLICATIONS

The present application is a continuation-in-part application of U.S. patent application Ser. No. 13/818,234, filed Feb. 21, 2013, which is a U.S. National Phase application of PCT Patent Application No. PCT/KR2011/006109, filed Aug. 19, 2011, which claims benefit of Korean, Republic of Patent Application Serial No. 10-2010-0081640, filed Aug. 23, 2010.

### BACKGROUND OF THE INVENTION

The present invention relates to a pharmaceutical composition for the prevention or treatment of immune disorders and inflammatory diseases, comprising stem cells that are generated by culturing stem cells expressing Nucleotide-binding Oligomerization Domain protein 2 (NOD2) with a NOD2 agonist or a culture thereof. More particularly, the present invention relates to a method for suppressing immune responses or inflammatory responses of a subject, comprising the step of administering the pharmaceutical composition, the stem cells or culture thereof to the subject, a method for preparing an immunosuppressive drug or an anti-inflammatory drug using the stem cells or culture thereof, a method for preparing PGE₂ or TGF-β1 comprising the step of culturing NOD2-expressing stem cells in culture medium with a NOD2 agonist, a graft comprising stem cells expressing NOD2 and the NOD2 agonist, a method for preparing the graft, a composite comprising stem cells expressing NOD2 and the NOD2 agonist, and a culture generated by culturing the NOD2-expressing stem cells with a NOD2 agonist.

### DESCRIPTION OF THE RELATED ART

Proteins that belong to a Nucleotide-binding Oligomerization Domain (NOD) protein family are composed of three main domains: a caspase-recruitment domain (CARD) or pyrin domain at N-terminal which is involved in a protein-protein interaction, a central NOD domain, and a LRR domain at C-terminal. Types of NOD proteins can be divided into a NOD1 group which has one CARD domain at N-terminal and a NOD2 group which has two CARD domains at N-terminal. These two groups are together called NOD-like receptor (NLR), and they contribute significantly to the development of immune response in vivo, along with a Toll-like receptor (TLR). NOD is known to play a vital role in an innate immune response by recognizing PGN moiety from the cell walls of most bacteria. NOD1 is expressed in epithelial cells of stomach and colon and in macrophages and dendritic cells of pancreas, lungs, kidney, and spleen. NOD1 is known to recognize a diaminopimelic acid (DAP)-type PGN stem peptide, dipeptide or tripeptide, which is present in gram-negative bacteria and a few gram positive bacteria, but absent in eukaryotes (Hisamatsu T et al., J. Biol. Chem., 278:32962, 2003).

NOD2 is expressed predominantly in myeloid cells, particularly macrophages, neutrophils, and dendritic cells, as well as in Paneth cells in the small intestine, and its distribution is more restricted than NOD1. Furthermore, the expression of NOD2 is induced by the inflammatory cytokines, i.e., TNF-alpha and IFN-gamma. As a result, NOD2 is barely expressed in the normal enterocytes, but is expressed only when the enterocytes are infected. The most widely known agonist (ligand) among the ones that are recognized by NOD2 is a muramyl dipeptide (MDP), which is the peptidoglycan (PGN) motif common to both of gram-negative and gram-positive bacteria (Girardin S E, et al., J. Biol. Chem., 278:8869, 2003).

In 2003, Girardin, S. E. et al. reported that NOD2-knockout mice can grow normally, but became highly sensitive to an infection by oral, but not intravenous, administration of Listeria monocytogenes. This result suggests that NOD2 is not involved in a growth mechanism but instead serves as a pattern recognition protein sensing the presence of MDP. However, in the presence of Listeria strain in intestine, NOD2 induces an innate immune response of antibacterial activity acting as a defense protein in the body (Girardin S E, et al., J. Biol. Chem., 278:8869, 2003). MDP has been used as an adjuvant for stimulating immune responses, i.e. antigenic adjuvant, and as an adjuvant capable of assisting an immunogen (Korean Patent Application Publication Nos. 1019960033469, 1020070031848, and 1020100045473).

Meanwhile, prostaglandin E2 (hereinafter, referred to as PGE₂) is a compound represented by prostaglandin E2: (5Z, 11(alpha), 13E, 15S)-11, 15-dihydroxy-9-oxo-prosta-5, 13-dien-1-oic acid, and is the most widely produced prostaglandin in physiological and pathological conditions (Ushikubi F et al., J. Pharmacol. Sci. 83:279, 2000).

PGE₂ was traditionally used to prepare the cervix for labor and has been actually used in pharmaceutical composition for stimulating childbirth. It is manufactured in a form of vaginal suppository with the following brand names: Cervidil (by Forest Laboratories, Inc.), Prostin E2 (by Pfizer Inc.), Propess (by Ferring Pharmaceuticals) and Glandin (by Nabiqasim Pharmaceuticals Pakistan). Recently, PGE₂ has been suggested as a strong candidate for new immunosuppressive modulator, as it functions to suppress release of cytokines such as interleukin-1 beta and TNF alpha which are produced by macrophage and also to suppress helper T1 cell differentiation (Harris S G et al., Trends Immunol., 23:144, 2002). Furthermore, in vitro study has reported that PGE₂ inhibits production of cytokines such as interleukin-2 and IFN-gamma so as to suppress human and murine T cell differentiation (Goodwin J S et al., J. clin. Immunol., 3:295, 1983). These studies suggest PGE₂ as a promising immunomodulatory drug, and as a result there has been a high need for development of a cost-effective and simple production method thereof.

Likewise, transforming growth factor beta 1 (TGF-β1) is known as an immunosuppressive and anti-inflammatory drug. TGF-β1, like PGE_{2,} has been suggested as a promising immunomodulatory drug, and as a result there has been a high need for development of a cost-effective and simple production method thereof.

Meanwhile, the types of immunosuppressive drugs can be divided into specific and non-specific immunosuppressants. Theoretically, specific immunosuppressants are superior, but non-specific immunosuppressants are mainly used. Cyclosporine (Neoral, Cipol A), Azathioprine (imuran), and Prednisolone (a steroid) are the most frequently used immunosuppressive drugs in clinical practice. It was found that a combination of these three drugs showed fewer side effects and higher immunosuppressive effects than use of individual drug. Recently, many immunosuppressive drugs such as FK 506, RATG; OKT3, Cellcept, etc. have been developed and used in clinical practice.

In the process from antigenic stimulation to antibody production, these immunosuppressive drugs cause immunosuppression by hindering phagocytic processing of antigens by macrophages, antigen recognition by lymphocytes, cell division, division of T and B cells, or antibody production. Most of the drugs have an antitumor activity, as they hinder cell division by inducing DNA injury, inhibition of DNA synthesis and the like.

However, the representative side effects of the drugs are hypertension and nephrotoxicity (reduction in renal function). Due to high occurrence of these side effects, the conditions of the patients had to be monitored adequately to detect the occurrence of the side effects. Side effects such as tremor, convulsion, hepatitis, cholestasis, hyperuricemia, muscle weakness, hypertrichosis, and gingival hypertrophy arise rarely. One of the frequently used suppressants called azathioprine suppresses bone marrow function incurring low leukocyte count, anemia, and low platelet count. In addition, azathioprine may cause complications such as pancreatitis, hepatitis, and cholestasis, as well as hair loss and fever occasionally. A steroid drug called prednisolone is the first immunosuppressant used in the market and has a variety of suppressive activity. For instance, it can increase one's appetite, the amount of muscle around the shoulder and the back, and can cause temporary euphoria. However, this steroid drug should be carefully used, since it promotes the progression of atherosclerosis, and causes hypertension, gastric ulcer, diabetes, growth retardation, osteoporosis, cataract, or glaucoma.

Allogeneic transplantation such as organ transplantation and hematopoietic stem cell transplantation is a remarkable medical achievement in the 21st century, and has been applied for radical treatment of terminal diseases such as heart failure including dilated cardiomyopathy, chronic renal failure, and intractable hematological disorders. However, there is still a limitation to overcome lethal complications arising after allogeneic transplantation, such as engraftment failure or graft-versus-host-disease (GVHD). In an effort to minimize these immune responses, a therapy for controlling T cell immune responses has been used which are caused by cellular immunity by T cells recognizing allogenic antigens after transplantation (Ikehara S, Exp. Hematol., 31:1142, 2003; First M R, Transplantation, 77:88, 2004), that is, a therapy of controlling immune responses by suppressing interleukin (IL)-2 production of T cell using an immunosuppressive drug, Cyclosporine or FK506. However, there is still a high demand for the development of inexpensive immunosuppressive drugs with no side effects.

Meanwhile, the immune regulation mechanisms of mesenchymal stem cells have not been fully identified yet, while only a few studies have been reported regarding mesenchymal stem cells. The first finding was that mesenchymal stem cells appear to suppress antigen presenting cell (APC). Changes in immune responses are proportional to the number of monocytes added during the culturing process under certain conditions, suggesting that monocytes are involved in immune suppression. The second finding was that mesenchymal stem cells appear to induce immunosuppressive properties by regulating T cell proliferation. Co-culturing of T cells with mesenchymal stem cells down-regulates the expression of cyclin D2 and subsequently arrests T cells in the G0/G1 phase of the cell cycle to prevent their proliferation. It was also reported that the proliferating ability is continuously reduced, even after mesenchymal stem cells are removed (Glennie S et al., Blood, 105:2821, 2005).

In an effort to develop more effective way of regulating immune or inflammatory responses using stem cells, the present inventors first found that NOD2 receptor is expressed in stem cells, and the stem cells regulate immune responses via NOD2 receptor. Then they discovered that when the stem cells are treated with a NOD2 agonist i.e. MDP, PGE₂ and TGF-β1 are excessively expressed, leading to more effective suppression of the immune response, thereby demonstrating its therapeutic effects on colitis and atopic dermatitis models completed the present invention.

### SUMMARY OF THE INVENTION

An object of the present invention is to provide a pharmaceutical composition for the prevention or treatment of immune disorders or inflammatory diseases, comprising stem cells that are generated by culturing stem cells expressing NOD2 with an NOD2 agonist or a culture thereof, which is inexpensive and has no side effect as an alternative to the conventional immunosuppressive drugs and anti-inflammatory drugs.

Another object of the present invention is to provide a method for treating immune disorders or inflammatory disease, comprising the step of administering the composition to a subject with immune disorders or inflammatory disease.

Still another object of the present invention is to provide a method for suppressing immune responses or inflammatory responses of a subject, comprising the step of administering the stem cells or the culture thereof to the subject.

Still another object of the present invention is to provide a method for preparing an immunosuppressive drug or anti-inflammatory drug.

Still another object of the present invention is to provide a method for preparing prostaglandin E₂ and TGF-β1 which are used in various applications.

Still another object of the present invention is to provide a graft comprising the stem cells expressing NOD2 and the NOD2 agonist, or a graft prepared by removing the stem cells from the graft, and a preparation method of the grafts.

Still another object of the present invention is to provide a composite comprising the stem cells expressing NOD2 and the NOD2 agonist.

Still another object of the present invention is to provide a culture that is generated by adding the NOD2 agonist to stem cells expressing NOD2.

### BRIEF DESCRIPTION OF THE DRAWINGS

FIG. 1a shows the mRNA RT-PCR result demonstrating TLR and NLR expression in hUCB-MSC, and FIGS. 1b to 1e show the level of IL-8 expression and MSC proliferation after treatment with each agonist;

FIG. 2 is a graph showing the effect of hUCB-MSC on MNC proliferation after treatment with each agonist;

FIG. 3 is a graph showing the effect of hUCB-MSC culture on MNC proliferation (3a and 3b and splenocyte proliferation (3c) after treatment with each agonist;

FIG. 4 shows the comparison of the inhibitory effect of mesenchymal stem cell (hUCB-MSC #618) culture treated with MDP alone (A) and the result of siRNA treatment (B);

FIG. 5 shows the comparison of the inhibitory effect of mesenchymal stem cell (hUCB-MSC #620) culture treated with MDP alone (A) and the result of siRNA treatment (B);

FIG. 6a shows the amount of PGE₂ secretion after treatment of each receptor with corresponding agonist, FIG. 6b shows the level of COX-2 expression after treatment of each receptor with corresponding agonist, FIG. 6c shows the changes in COX-2 expression level which is increased by treatment of siNOD2 and siRip2 with MDP, FIG. 6d shows the changes in the amount of PGE₂ secretion which is increased by treatment of siNOD2. siRip2 and indomethacin (indo) with MDP, FIG. 6e shows the changes in the inhibitory effect on MNC proliferation, which is increased by treatment of indomethacin (indo) with MDP, and FIG. 6f shows the reduction in the amount of PGE₂ secretion which is enhanced by treatment of siNOD2 and indomethacin (indo) with MDP;

FIG. 7a shows the amount of NO production after treatment of each receptor with corresponding agonist, FIG. 7b shows the amount of PGE₂ secretion after treatment of each receptor with corresponding agonist, FIG. 7c shows the amount of TGF-β1 secretion after treatment of each receptor with corresponding agonist, FIG. 7d shows change in the COX-2 expression level after treatment of each receptor with corresponding agonist, FIG. 7e shows inhibitory effect on MNC proliferation in the presence of PGE_{2,} FIG. 7f shows inhibitory effect on splenocyte proliferation in the presence of PGE_{2,} FIG. 7g shows inhibitory effect on MNC proliferation, which is enhanced by treatment of indomethacin (indo) with MDP, and FIG. 7h shows inhibitory effect on MNC proliferation, which is improved by treatment of TGF-β1 neutralizing antibody (a-TGF-β1) with MDP;

FIG. 8 demonstrates remarkable suppression of NOD2 and Rip2 genes and proteins expression in hUCB-MSC by respective siRNA;

FIG. 9a shows COX-2 expression increased by treatment of siNOD2 and siRip2 with MDP, FIG. 9b shows PGE₂ secretion increased by treatment of siNOD2, siRip2 and indomethacin (indo) with MDP, FIG. 9c shows TGF-β1 secretion increased by treatment of siNOD2 and siRip2 with MDP, and FIG. 9d is a graph showing the changes in inhibitory effect on MNC proliferation by treatment of siNOD2 and siRip2 with MDP;

FIG. 10a shows IL-10 secretion enhanced by treatment of siNOD2, siRip2, indomethacin (indo) and TFG-β1 neutralizing antibody (a-TFG-β1) with MDP, and FIG. 10b shows the changes in Treg populations which are enhanced by treatment of siNOD2, siRip2, indomethacin (indo) and TFG-β1 neutralizing antibody (a-TFG-β1) with MDP;

FIG. 11 shows mRNA RT-PCR result demonstrating NOD2, RIP2 and RPL13A expressions in umbilical cord blood-derived mesenchymal stem cells (UCB-MSC), adipose tissue-derived stem cells (AD-MSC) and amniotic epithelial stem cells (AEC);

FIG. 12a is a graph showing body weight reduction in experimental groups and control groups of colitis model, FIG. 12b is a graph showing survival rate, FIG. 12c is a graph showing the changes in disease activity index. FIG. 12d shows the image of colon length, FIG. 12e is a graph showing the colon length, FIG. 12f is a histopathological image showing inflammation, edema, and infiltration of inflammatory cells, and FIG. 12g is a graph showing pathological scores;

FIG. 13a is a graph showing changes in IL-6, IFN-γ and IL-10 secretion by MDP in the colon of DSS-induced colitis mouse model, FIG. 13b is a fluorescence microscopic image showing Fox3p+ localization in the colons of control groups and experimental groups of colitis mouse model after MDP and siNOD2 treatment, FIG. 13c is the result of Western blotting showing Fox3p protein expression level in control groups and experimental groups after MDP and siNOD2 treatment, FIG. 13d shows the quantification of the Western blot analysis, FIG. 13e is a graph demonstrating MPO activity and CD4+, CD11b+ cell infiltration for analyzing infiltration of inflammatory cells in mouse colon;

FIG. 14 is a graph showing the result of gross examination after intravenous or subcutaneous injections of MDP-treated hUCB-MSC into atopic dermatitis-induced mouse;

FIG. 15 is a graph showing a serum immunoglobulin E level, which is an index of atopic dermatitis;

FIG. 16 is a graph showing a serum immunoglobulin G1 level, which is an index of atopic dermatitis;

FIG. 17 is an image of H&E staining after tissue processing of the mouse skin tissue; and

FIG. 18 is an image showing mast cell degranulation, which is one of the major symptoms of atopic dermatitis, after tissue processing and Toluidine blue staining of the mouse skin tissue.

FIG. 19a is an image showing a survival rate and body weight loss, FIG. 19b shows measurement of colon length, and FIG. 19c shows a histopathologic evaluation of colon sections. Five mice per group were used, Bar, 500 mm. FIG. 19d is an image shown a percent survival and body weight after administration of NOD2-deficient hUCB-MSCs (CTL is a control). FIG. 19e is an image showing a body weight (%) of a mice at 9 days after colitis induction and hUCB-MSC administration.

FIG. 20 is an image showing a mechanism of NOD2 activation by bacterial muramyl dipeptide in an inflammatory bowel disease patient.

### DETAILED DESCRIPTION OF THE PREFERRED EMBODIMENTS

In one aspect to achieve the above objects, the present invention provides a pharmaceutical composition for the prevention or treatment of immune disorders or inflammatory diseases, comprising stem cells that are generated by culturing stem cells expressing Nucleotide-binding Oligomerization Domain protein 2 (NOD2) with a NOD2 agonist or a culture thereof.

In the present invention, it was found that the treatment of stem cells with Nucleotide-binding Oligomerization Domain protein 2 (NOD2) agonist promotes the secretion of PGE₂ and TGF-β1 in the mesenchymal stem cells, which in turn regulates immune and inflammatory responses. In other words, the present invention confirmed that immune and inflammation regulatory activity of stem cells are correlated with the function of NOD2, and immunosuppressive and anti-inflammatory effects are enhanced when treated with NOD2 agonist, and thus the NOD2 agonist-treated stem cells and a culture thereof can be used as a cellular therapeutic agent for immune and inflammation regulation. Therefore, the present invention provides a pharmaceutical composition for the prevention or treatment of immune disorders or inflammatory diseases, comprising stem cells that are generated by culturing stem cells expressing Nucleotide-binding Oligomerization Domain protein 2 (NOD2) with a NOD2 agonist or a culture thereof. As used herein, the terms ‘NOD2’ and ‘NOD2 receptor’ can be used interchangeably.

As used herein, the term ‘agonist’ generally refers to a chemical that functions to stimulate a receptor positively, and is also called an effector. In other words, an agonist has a positive function, while antagonist functions to hinder a ligand or has a negative function. In the present invention, the agonist can be used interchangeably with ‘ligand’ which refers to a chemical that binds to a receptor in general. With respect to the objects of the present invention, the agonist may be a NOD2 agonist.

As used herein, the term ‘NOD2 agonist’ refers to a substance that binds to a NOD2 receptor to activate NOD2, and one of the examples of NOD2 agonist is Muramyl Dipeptide (MDP) but is not limited thereto.

As used herein, the term ‘MDP’ is muramyl dipeptide, and in the present invention it can be used as an agonist that activates NOD2 pathway to promote secretion of PGE₂ in the mesenchymal stem cells.

As used herein, the phrases ‘cultured with addition’ or ‘generated with addition’ of agonist may refer to culturing of mesenchymal stem cells in a culture medium added with an agonist as an example. Preferably, the above culturing may refer to culturing with addition of the agonist at a concentration of 1 to 100 μg/ml for 0.1 to 200 hours, and more preferably for 1 to 72 hours. In addition, it may refer to culturing of the cells in the medium added with the agonist, and further culturing in the replaced medium.

As used herein, the term ‘stem cells’ refers to cells that have the capability to differentiate into various tissues, i.e., ‘undifferentiated cells’. The term ‘mesenchymal stem cells’ refers to pluripotent stem cells derived from various adult cells such as bone marrow, umbilical cord blood, placenta (or placental tissue) and fat (adipose tissue). For example, mesenchymal stem cells derived from bone marrow possess a pluripotency to differentiate into adipose tissue, bone/cartilage tissue, and muscle tissue and thus many studies have focused on investigating mesenchymal stem cells for the development of cell therapy.

In the present invention, the stem cells may be human adult stem cells, human pluripotent stem cells, induced pluripotent stem cells, animal embryonic stem cells or animal adult stem cells. Meanwhile, the adult stem cells may be mesenchymal stem cells, human tissue-derived mesenchymal stromal cell, human tissue-derived mesenchymal stem cells, pluripotent stem cells or amniotic epithelial cells, and the mesenchymal stem cells may be mesenchymal stem cells derived from a source selected from the group consisting of umbilical cord, umbilical cord blood, bone marrow, fat, muscle, nerve, skin, amnion and placenta, and preferably those derived from human, and most preferably mesenchymal stem cells (hUCB-MSCs) derived from human umbilical cord blood. Obtaining stem cells from each source may be performed following the method known in the art, and is not limited to the method described in Examples of the present invention.

Preferably, mesenchymal stem cells prepared by treatment of human-derived mesenchymal stem cells with MDP at a concentration of 1 to 100 μg/ml for 0.1-200 hours are used. If the cells are cultured with MDP for 0.1 hour or shorter, NOD2 pathway cannot be sufficiently activated. If the cells are cultured with MDP for 200 hours or longer, there are no financial benefits. Thus, mesenchymal stem cells are treated with MDP more preferably for 0.1˜200 hours, much more preferably for 1˜72 hours, and most preferably for 24 hours.

For culturing the mesenchymal stem cells, any conventional medium known in the art can be used that are known to be suitable for stem cell culturing. For example, Dulbecco's modified Eagle medium (DMEM) or Keratinocyte serum-free medium (Keratinocyte-SFM) may be used. Most preferably, D-media (Gibco) may be used.

The medium for culturing mesenchymal stem cells may be supplemented with additives. Generally, the medium may contain a neutral buffer (e.g., phosphate and/or high concentration bicarbonate) in isotonic solution and a protein nutrient (e.g., serum such as FBS, serum replacement, albumin, or essential and non-essential amino acids such as glutamine). Furthermore, it may contain lipids (fatty acids, cholesterol, an HDL or LDL extract of serum) and other ingredients found in most stock media of this kind (e.g., insulin or transferrin, nucleosides or nucleotides, pyruvate, a sugar source such as glucose, selenium in any ionized form or salt, a glucocorticoid such as hydrocortisone and/or a reducing agent such as (β-mercaptoethanol).

Also, with a view to protecting cells from adhering to each other or to a vessel wall, or from forming large clusters, it may be beneficial to include an anti-clumping agent in the medium, for example, those sold by Invitrogen (Cat # 0010057AE).

In one embodiment of the present invention, it was found that the culture of stem cells which were cultured with addition of one of NOD2 agonists, Muramyl Dipeptide (MDP) inhibits proliferation of mononuclear cells (MNC).

Mononuclear cells circulating in bloodstream migrate to tissues where they mature into macrophages. Mononuclear cells, macrophages, and dendritic cells are the most important ones in the body defense system, and have a central role in the initiation of adaptive immune responses having the ability to present antigen and regulate the function of T-lymphocyte. On the other hand, mononuclear cells and macrophage act as the primary defense barriers in immune system. Also, mononuclear cells function as accessory cells in the recognition and activation steps of adaptive immune responses. They function as antigen presenting cells (APCs) for antigen recognition by T-lymphocytes, and produce membrane proteins and secretory proteins that function as secondary signals for T cell activation. Some of mononuclear phagocytes can differentiate into dendritic cells, which play an important role in stimulation of T lymphocyte responses against protein antigens. When cell and organ transplant rejection occurs, the cell/organ transplanted in the body is recognized as a foreign object, and therefore, the number of mononuclear cells, macrophages, and dendritic cells all increase. Thus, it is apparent to those skilled in the art that suppression of the mononuclear cell proliferation by using the culture of stem cells generated with addition of Muramyl Dipeptide (MDP) leads to suppression of immune responses in the body.

As used herein, the term ‘mononuclear cell’ refers to a mononuclear phagocytic leukocyte derived from the bone marrow and peripheral blood cells.

Furthermore, in another embodiment of the present invention, it was found that stem cells cultured with addition of Muramyl Dipeptide (MDP) promotes secretion of PGE₂ and TGF-β1, leading to MNC suppression that is induced by PGE₂ and TGF-β1. It has been reported that PGE₂ functions to inhibit the secretion of inflammatory cytokines such as interleukin-1 beta and cytokines TNF alpha. TGF-β1 is considered as an anti-inflammatory cytokine.

In still another embodiment of the present invention, it was suggested that MDP-treated stem cells produce an anti-inflammatory cytokine IL-10 at high yield, and furthermore, forms a regulatory T cell population.

Therefore, the stem cells of the present invention and the culture thereof are useful for the prevention or treatment of immune disorders and inflammatory diseases. In this regard, the immune disorders or inflammatory diseases may be autoimmune diseases, transplant rejection, graft-versus-host-disease, arthritis, bacterial infection, sepsis, inflammation or the like. The autoimmune diseases may be Crohn's disease, erythema, atopic dermatitis, rheumatoid arthritis, Hashimoto's thyroiditis, pernicious anemia, Addison's disease, type 1 diabetes, lupus, chronic fatigue syndrome, fibromyalgia, hypothyroidism and hyperthyroidism, scleroderma, Behcet's disease, inflammatory bowel disease, multiple sclerosis, myasthenia gravis, Meniere's syndrome, Guillain-Barre syndrome, Sjogren's syndrome, vitiligo, endometriosis, psoriasis, vitiligo, systemic scleroderma, asthma, ulcerative colitis or the like.

As used herein, the term ‘inflammatory diseases’ collectively mean lesions caused by inflammation, and may be, but not limited to, preferably edema, dermatitis, allergy, atopic dermatitis, asthma, conjunctivitis, periodontitis, rhinitis, tympanitis, pharyngolaryngitis, amygdalitis, pneumonia, gastric ulcer, gastritis, Crohn's disease, colitis, haemorrhoids, gout, ankylosing spondylitis, rheumatic fever, lupus, fibromyalgia, psoriatic arthritis, osteoarthritis, rheumatoid arthritis, Periarthritis of shoulder, tendonitis, tenosynovitis, myositis, hepatitis, cystitis, nephritis, sjogren's syndrome or multiple sclerosis.

As used herein, the term ‘immune disorders’ refers to the disorders that are associated with the development of particular immune responses, and may be, but not limited to, preferably autoimmune diseases, transplant rejection, graft-versus-host-disease. The autoimmune diseases may be Crohn's disease, erythema, atopic dermatitis, rheumatoid arthritis, Hashimoto's thyroiditis, pernicious anemia, Addison's disease, type 1 diabetes, lupus, chronic fatigue syndrome, fibromyalgia, hypothyroidism and hyperthyroidism, scleroderma, Behcet's disease, inflammatory bowel disease, multiple sclerosis, myasthenia gravis, Meniere's syndrome, Guillain-Barre syndrome, Sjogren's syndrome, vitiligo, endometriosis, psoriasis, vitiligo, systemic scleroderma, asthma, ulcerative colitis or the like.

In the embodiments of the present invention, it was confirmed that the stem cells of the present invention or the culture thereof could treat the inflammatory disease such as colitis and immune disease such as atopic dermatitis in colitis animal models and atopic dermatitis models, suggesting its therapeutic effects on immune disorders and inflammatory diseases.

As used herein, the term ‘prevention’ means all of the actions in which immune disorders or inflammatory diseases are restrained or retarded by the administration of the composition. As used herein, the term ‘treatment’ means all of the actions in which the symptoms of immune disorders or inflammatory diseases are relieved or turned into better condition by the administration of the composition.

Furthermore, the composition of the present invention may include 1.0×105 to 1.0×109, preferably 1.0×106 to 1.0×108, more preferably 1.0×107 cells per 1 ml.

The composition of the present invention may be used unfrozen, or frozen for later use. If the population of cells is to be frozen, a standard cryopreservative (e.g., DMSO, glycerol, Epilife® Cell Freezing Medium (Cascade Biologics) is added to the enriched population of cells before it gets frozen.

Furthermore, the composition may be administered by formulating a unit dosage suitable for administering to a patient by conventional methods in the pharmaceutical field, with the formulation containing an effective amount for a single dose or for divided doses. For this purpose, a formulation for parenteral administration preferably includes an injection formulation such as injection ampoule, an infusion formulation such as infusion bag, and a spray formulation such as aerosol. The injection ampoule may be mixed with an injection solution such as saline solution, glucose, mannitol and ringer solution immediately before administration of the formulation. Furthermore, the infusion bag may be textured with polyvinyl chloride or polyethylene, for example, a product of Baxter, Becton Dickinson, Medcep, National Hospital Products or Terumo.

The pharmaceutical formulation may further comprise one or more pharmaceutically acceptable inactive carriers, for example, a preservative, an analgesic controller, a solubilizer or a stabilizer for injection formulation, and a base, an excipient, a lubricant or a preservative for topical formulation, in addition to the active ingredient.

The prepared composition or pharmaceutical formulation of the present invention may be administered in accordance with any conventional method in the art together with other stem cells used for transplantation and other purposes, or in the form of a mixture therewith. Direct engraftment or transplantation to the lesion of a patient in need of treatment, or direct transplantation or injection into the peritoneal cavity is preferred, but is not limited thereto. Furthermore, both of a non-surgical administration using a catheter and a surgical administration such as injection or transplantation after incision are possible, but non-surgical administration using a catheter is more preferred. In addition, the composition can also be administered parenterally, for example, intravenous injection, which is one of the conventional methods for transplantation of stem cells of hematopoietic system, besides direct administration to the lesion.

The stem cells may be administered in an amount of 1.0×104 to 1.0×10¹⁰ cells/kg (body weight), preferably 1.0×10⁵ to 1.0×10⁹ cells/kg (body weight) per day in a single dose or in divided doses. However, it should be understood that the amount of the active ingredient actually administered ought to be determined in light of various relevant factors including the disease to be treated, the condition to be treated, the severity of the patient's symptom, the chosen route of administration, and the body weight, age and sex of the individual patient; and, therefore, the above dose should not limit the scope of the invention in any way.

In another aspect, the present invention provides a method for treating immune disorders or inflammatory disease, comprising the step of administering the composition to a subject with immune disorder or inflammatory disease.

In still another aspect, immune responses can be suppressed or inflammation can be regulated by administration of the NOD2 agonist-treated stem cells according to the present invention and the culture thereof, and therefore, the present invention provides a method for suppressing immune responses or inflammatory responses of a subject, which involves the step of administering the stem cells generated by adding the NOD2 agonist to stem cells expressing NOD2 or the culture thereof to the subject.

As used herein, the term ‘subject’ means a mammal including cattle, dogs, swine, chickens, sheep, horses, and human, but is not limited thereto. In this regard, the method for suppressing immune responses or inflammatory responses may be limited to animals excluding human. Preferably, administration of the stem cells cultured with addition of NOD2 agonist or the culture thereof may be performed by intra-abdominal, intraarterial injection, intravenous injection, direct injection into the lesion, or injection into the synovial cavity.

The suppression of immune responses or inflammatory responses is for prevention or treatment of immune disorders or inflammatory diseases.

In still another aspect, the present invention provides a method for preparing an immunosuppressive drug or an anti-inflammatory drug, which involves the step of culturing stem cells by adding NOD2 agonist to stem cells expressing Nucleotide-binding Oligomerization Domain protein 2 (NOD2).

As used herein, the term ‘immunosuppressive drug’, as described above, means a drug comprising stem cells generated by culturing stem cells expressing NOD2 with the NOD2 agonist or the culture thereof, which is able to treat immune disorders by suppressing immune responses.

As used herein, the term ‘anti-inflammatory drug’, as described above, means a drug comprising stem cells generated by culturing stem cells expressing NOD2 with the NOD2 agonist or the culture thereof, which is able to treat inflammatory diseases by suppressing inflammation.

In still another aspect, the present invention provides a method for preparing PGE₂ or TGF-β1, which involves the step of culturing stem cells expressing Nucleotide-binding Oligomerization Domain protein 2 (NOD2) in a medium treated with NOD2 agonist, in which prostaglandin E2 (PGE₂) or transforming growth factor beta 1 (TGF-β1) is secreted from the stem cells during culturing.

In the embodiments of the present invention, MDP-treated mesenchymal stem cells were found to promote secretion of PGE₂ and TGF-β1 significantly which are known to be applicable in various fields, as compared to mesenchymal stem cells untreated with MDP and mesenchymal stem cells treated with other receptor agonists (DAP, LPS, Pam3CSK4, etc.).

In the present invention, for recovery of PGE₂ and TGF-β1, the culture medium of the stem cells is collected, and cells and debris are removed by centrifugation and filtration, thereby leaving the supernatant only.

In still another aspect, the present invention provides a graft comprising the stem cells expressing Nucleotide-binding Oligomerization Domain protein 2 (NOD2) and the NOD2 agonist.

As used herein, the term ‘graft’ means a material that can be transplanted into human or mammal, which protects the damaged tissue from outside or supports a transplanted cell or secreted therapeutic substance to remain in the same place. The graft is used as a support for tissue engineering and involves biodegradable synthetic polymers and natural materials that are used in the art but is not limited thereto. Since the graft of the present invention comprises the stem cells expressing NOD2 and the NOD2 agonist, there is an advantage in that it does not cause transplant rejection or inflammatory responses. Therefore, without any additional immunosuppressive agent or anti-inflammatory drug needed to suppress transplant rejection or inflammatory responses caused by transplantation of various grafts, the transplanted graft can be stably engrafted on the body without incurring transplant rejection or inflammatory responses.

In still another aspect, the present invention provides a graft that is prepared by culturing the stem cells expressing Nucleotide-binding Oligomerization Domain protein 2 (NOD2) in the graft with a NOD2 agonist, and then removing stem cells therefrom.

In still another aspect, the present invention provides a method for preparing the graft, comprising the step of culturing the stem cells expressing Nucleotide-binding Oligomerization Domain protein 2 (NOD2) in the graft with a NOD2 agonist.

Preferably, the method may further comprises the step of removing stem cells after the culturing step.

In still another aspect, the present invention provides a composite comprising the stem cells expressing Nucleotide-binding Oligomerization Domain protein 2 (NOD2) and the NOD2 agonist.

Preferably, the NOD agonist may bind to NOD2 of the stem cells in the composite. More preferably, NOD2 may be activated by binding of the NOD2 agonist to NOD2 of the stem cells in the composite. Ultimately, the composite may be used for cell therapy.

In still another aspect, the present invention provides a culture that is generated by culturing stem cells expressing Nucleotide-binding Oligomerization Domain protein 2 (NOD2) with a NOD2 agonist.

The culture may comprise components such as PGE₂ and/or TGF-β1, which exhibit the prophylactic or therapeutic effects on immune disorders or inflammatory diseases.

In still another aspect, the present invention provides a method for treatment of immune diseases or inflammatory diseases, comprising steps of (a) preparing isolated stem cells in which the expression of NOD2 is determined; and (b) administering the stem cells of step (a) or a culture thereof to the subject.

Activation of NOD2 is required for the ability of hUCB-MSCs to treat immune diseases or inflammatory diseases. NOD2 signaling activated by a NOD2 agonist existed in the body of a patient increases the ability of these stem cells to suppress mononuclear cell proliferation by inducing production of PGE₂.

In this regard, the immune disorders diseases or inflammatory diseases may be autoimmune diseases, transplant rejection, arthritis, graft-versus-host-disease, bacterial infection, sepsis or inflammation.

Specifically, the autoimmune diseases may be selected from the group consisting of Crohn's disease, erythema, atopic dermatitis, rheumatoid arthritis, Hashimoto's thyroiditis, pernicious anemia, Addison's disease, type 1 diabetes, lupus, chronic fatigue syndrome, fibromyalgia, hypothyroidism and hyperthyroidism, scleroderma, Behcet's disease, inflammatory bowel disease, multiple sclerosis, myasthenia gravis, Meniere's syndrome, Guilian-Barre syndrome, Sjogren's syndrome, vitiligo, endometriosis, psoriasis, vitiligo, systemic scleroderma, asthma, and ulcerative colitis.

The stem cells may be human adult stem cells, human pluripotent stem cells, induced pluripotent stem cells, animal embryonic stem cells or animal adult stem cells.

Specifically, the adult stem cells may be mesenchymal stem cells, human tissue-derived mesenchymal stromal cell, human tissue-derived mesenchymal stem cells, pluripotent stem cells or amniotic epithelial cells.

Specifically, the adult stem cells may be mesenchymal stem cells selected from the group consisting of umbilical cord-derived mesenchymal stem cells, umbilical cord blood-derived mesenchymal stem cells, bone marrow-derived mesenchymal stem cells, adipose tissue-derived mesenchymal stem cells, muscle-derived mesenchymal stem cells, nerve-derived mesenchymal stem cells, skin-derived mesenchymal stem cells, amnion-derived mesenchymal stem cells, and placenta-derived mesenchymal stem cells.

In one embodiment of the present invention, the step may be performed by intra-abdominal, intraarterial injection, intravenous injection, direct injection into the lesion, or injection into the synovial cavity.

In still another aspect, the present invention provides a pharmaceutical composition for the prevention or treatment of immune diseases or inflammatory diseases, comprising isolated mesenchymal stem cells expressing Nucleotide-binding Oligomerization Domain protein 2 (NOD2) or a culture thereof.

In this regard, the expression of NOD2 may be determined in the stem cells.

In one embodiment of the present invention, the pharmaceutical composition may further comprise a NOD2 agonist. Specifically, the NOD2 agonist may be muramyl dipeptide (MDP).

In still another aspect, the present invention provides a graft that is prepared by determining expression of Nucleotide-binding Oligomerization Domain protein 2 (NOD2) in isolated mesenchymal stem cells, and culturing the stem cells expressing NOD2 on the graft support.

In this regard, the graft may further comprise a NOD2 agonist. Specifically, the NOD2 agonist may be muramyl dipeptide (MDP).

In still another aspect, the present invention provides a method for preparing a graft, comprising the steps of (a) determining expression of Nucleotide-binding Oligomerization Domain protein 2 (NOD2) in isolated mesenchymal stem cells; and (b) culturing the stem cells of step (a) in the graft.

In one embodiment of the present invention, the stem cells of step (b) may be cultured with a NOD2 agonist. Specifically, the NOD2 agonist may be muramyl dipeptide (MDP).

In one embodiment of the present invention, the method may further comprise the step of removing stem cells after the culturing step.

Hereinafter, the present invention is described in more detail through providing Examples as below. However, these Examples are merely meant to illustrate, but in no way to limit, the claimed invention.

In the following Examples, only the use of mesenchymal stem cells derived from umbilical cord blood is exemplified, but it is apparent to those skilled in the art from the foregoing description that the mesenchymal stem cells and stem cells having other NOD2 receptors can be treated with NOD2 agonists for inducing a remarkably increase in the level of PGE₂ production in order to get immunosuppressive or anti-inflammatory effects.

Moreover, MDP was used as a NOD2 agonist in the present Examples. However, as shown in the following Examples, the stem cells wherein NOD2 receptors are inhibited have no immune regulatory activity, and thus it will be apparent to those skilled in the art from the foregoing description that even when other NOD2 agonists are used, the immunosuppressive or anti-inflammatory effects of the present invention can be achieved.

### EXAMPLE 1

### Isolation and Culturing of Human Umbilical Cord Blood-Derived Mesenchymal Stem Cells (Hereinafter, Referred to as hUCB-MSC) and Human Umbilical Cord Blood-Derived Mononuclear Cells (Hereinafter, Referred to as hUCB-MNC)

The Umbilical Cord Blood (UCB) samples were obtained from the umbilical vein immediately after delivery, with the written consent of the mother approved by the Boramae Hospital and the Seoul National University Institutional Review Board (IRB No. 0603/001-002-07C1). The UCB samples were mixed with the Hetasep solution (StemCell Technologies, Vancouver, Canada) in a ratio of 5:1, and then incubated at room temperature to remove erythrocyte. The mononuclear cells were carefully collected by adding Ficoll solution to the sample and centrifuging the mixture at 2500 rpm for 20 minutes separating it from the supernatant. Then the pelleted cells were washed twice with PBS.

The hUCB-derived mononuclear cells (hUCB-MNCs) were cultured in RPMI-1640 medium (Gibco, Grand Island, N.Y., USA) supplemented with 10% fetal bovine serum (FBS).

The hUCB-derived mesenchymal stem cells (hUCB-MSCs) were cultured at a density of 2×10⁵˜2×10⁶ cells/cm2 in D-media (Formula No. 78-5470EF, Gibco BRL) which contains EGM-2 SingleQuot and 10% FBS (Gibco BRL). After 3 days of culturing, non-adherent cells were removed. It was observed that the adherent cells formed colonies and grew rapidly, showing spindle-shaped morphology. The mesenchymal stem cells isolated from each of the UCB sample were designated as #618 and #620 respectively.

### EXAMPLE 2

### Identification of the Receptors Expressed in hUCB-MSC

2-1: Identification of the Expression of Functional TLR2, TLR4, NOD1, and NOD2 in hUCB-MSC

RT-PCR was performed to determine whether functional Toll Like Receptor 2 (TLR2), Toll Like Receptor 4 (TLR4), Nucleotide-binding Oligomerization Domain proteins 1 (NOD1) and Nucleotide-binding Oligomerization Domain proteins 2 (NOD2) are expressed in hUCB-MSCs.

To be specific, total RNA was extracted from hUCB-MSCs by using an Easy-spin total RNA extraction kit (Intron Biotechnology, Seongnam, Korea). cDNA was prepared from 1 μg of total RNA by using Superscript III reverse transcriptase (Invitrogen, Carlsbad, Calif., USA) and oligo (dT) primers (Invitrogen). The primer sets used are as follows (F: Forward, R: Reverse).

TLR2 F (SEQ ID NO. 1):
5′-GATGCCTACTGGGTGGAGAA-3′
TLR2 R (SEQ ID NO. 2):
5′-CGCAGCTCTCAGATTTACCC-3′
TLR4 F (SEQ ID NO. 3):
5′-ACAGAAGCTGGTGGCTGTG-3′
TLR4 R (SEQ ID NO. 4):
5′-TCTTTAAATGCACCTGGTTGG-3′
NOD1 F (SEQ ID NO. 5):
5′-CCACTTCACAGCTGGAGACA-3′
NOD1 R (SEQ ID NO. 6):
5′-TGAGTGGAAGCAGCATTTTG-3′
NOD2 F (SEQ ID NO. 7):
5′-GAATGTTGGGCACCTCAAGT-3′
NOD2 R (SEQ ID NO. 8):
5′-CAAGGAGCTTAGCCATGGAG-3′
Rip2 F (SEQ ID NO. 9):
5′-CCATTGAGATTTCGCATCCT-3′
Rip2 R (SEQ ID NO. 10):
5′-ATGCGCCACTTTGATAAACC-3′
RPL13A F (SEQ ID NO. 11):
5′-CATCGTGGCTAAACAGGTAC-3′
RPL13A R (SEQ ID NO. 12):
5′-GCACGACCTTGAGGGCAGCC-3′

The PCR condition was set to have an initial denaturation at 95° C. for 3 min; 30 cycles of 94° C. for 30 sec, 60° C. for 30 sec and 72° C. for 1 min; a final extension at 72° C. for 10 min. The PCR products were separated on a 1.5% agarose gel, visualized, and the image of the gel was photographed using a gel documentation system.

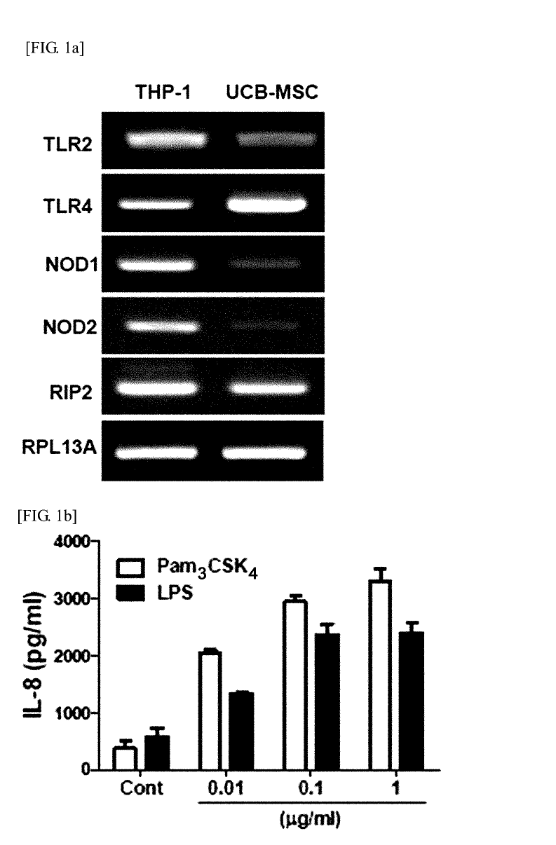

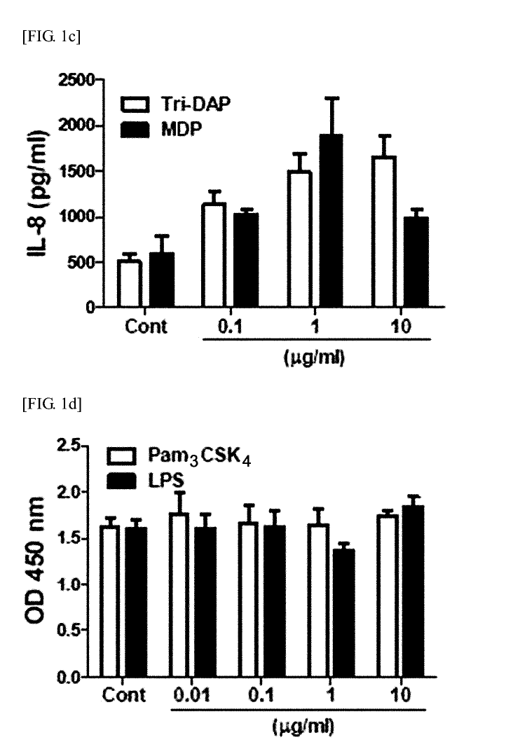

As shown in FIG. 1a, in a positive control group containing a human monocytic leukemia cell line, i.e. THP-1 cell, all of the receptors of interest were expressed in both THP-1 cells and hUCB-MSCs. TLR4 was expressed at higher level in hUCB-MSCs than in THP-1 cells, whereas the gene expression levels of TLR2, NOD1, and NOD2 were greater in THP-1 cells. Meanwhile, Rip2 expression was also observed in hUCB-MSCs.

2-2: Analysis of Cytokine Production in Response to Stimulation by Agonists of the Receptors of Interest.

After confirming the expression of the receptors of interest in hUCB-MSC in Example 2-1, the functionality of the receptors were investigated by monitoring IL-8 production after stimulation by agonists. For this experiment, hUCB-MSCs were cultured at a density of 2×10⁴ cells/well in KSFM medium supplemented with 2% FBS in a 96-well plate. After 24 hours of culturing, the cells were treated with the following agonists corresponding to each of the receptors, i.e., Pam3CSK4 (TLR2 agonist, Pam3), LPS (TLR4 agonist), Tri-DAP (NOD1 agonist, T-DAP), and MDP (NOD2 agonist). Then the samples were incubated for additional 24 hours. The supernatant of each culture was collected, centrifuged, and filtered through a 0.2 μm filter. Then, concentrations of IL-8 and PGE₂ were measured using an ELISA kit (R&D Systems, Minneapolis, Minn., USA). Ultrapure LPS (E. coli O111:B4), Pam3CSK4, and Tri-DAP were purchased from Invivogen (San Diego, Calif., USA). MDP [Ac-(6-O-stearoyl)-muramyl-Ala-D-Glu-NH2; muramyl dipeptide] was purchased from Bachem (Bubendorf, Switzerland). Recombinant human Interferon-γ was purchased from Peprotech (Rockyhill, N.J., US

As shown in FIGS. 1b and 1c, stimulation by Pam3CSK4 (Tri-acylated peptide; TLR2 agonist), LPS (Lipopolysaccharide, TLR4 agonist), Tri-DAP (Tri-diaminopimelic Acid, NOD1 agonist), and MDP (NOD2 agonist) led to the increased IL-8 production in hUCB-MSCs in a dose-dependent manner. These results suggest that NOD1, NOD2, TLR2 and TLR4 are expressed and actively respond to the stimulation by agonists in hUCB-MSCs.

2-3: Analysis of the Effects of TLR and NLR Stimulation by Agonists on hUCB-MSC Proliferation (1)

Based on the previous finding that a certain type of MSC is affected by TLR stimulation (Pavsner-Ficher et al., Toll-like receptors and their ligands control mesenchymal stem cell functions, Blood, 109:1422, 2007), the effect of agonists on hUCB-MSC proliferation was investigated by treating the cells with each of the agonists, and culturing them for 4 days.

More particularly, cells were cultured at a density of 2×103 cells/well in MSC medium supplemented with 2% FBS in a 96-well plate. After 24 hours of culturing, the cells were treated with Pam3CSK4 (TLR2 agonist), LPS (TLR4 agonist), Tri-DAP (NOD1 agonist), and MDP (NOD2 agonist) at a concentration of 10 μg/ml each and then cultured for 4 more days. Cell proliferation was monitored by using Cell Counting Kit-8 (Dojindo Molecular Technologies, Rockville, Md., USA). The difference in results for each type of experimental groups was represented by standard deviation (±SD). All statistical analysis was performed using MS Excel program, and test values of p<0.05 were regarded as statistically significant (hereinafter, the same).

As shown in FIGS. 1d and 1e, none of the agonists were found to have an effect on proliferation of hUCB-MSC.

2-4: Analysis of the Effects of TLR and NLR Stimulation by Agonists on the Suppressive Activity of hUCB-MSC Against Human MNC Proliferation (2)

In the present experiment, the inventors investigated whether TLR and NLR agonists enhance the suppressive activity of hUCB-MSC against human MNC proliferation.

Based on the study that identified the importance of a direct interaction between MSCs and lymphocytes in inhibition of lymphocyte proliferation by MSCs, the present inventors investigated whether MDP has an effect on the suppressive ability of hUCB-MSCs against MNC proliferation under the conditions where two cell groups are in contact with each other.

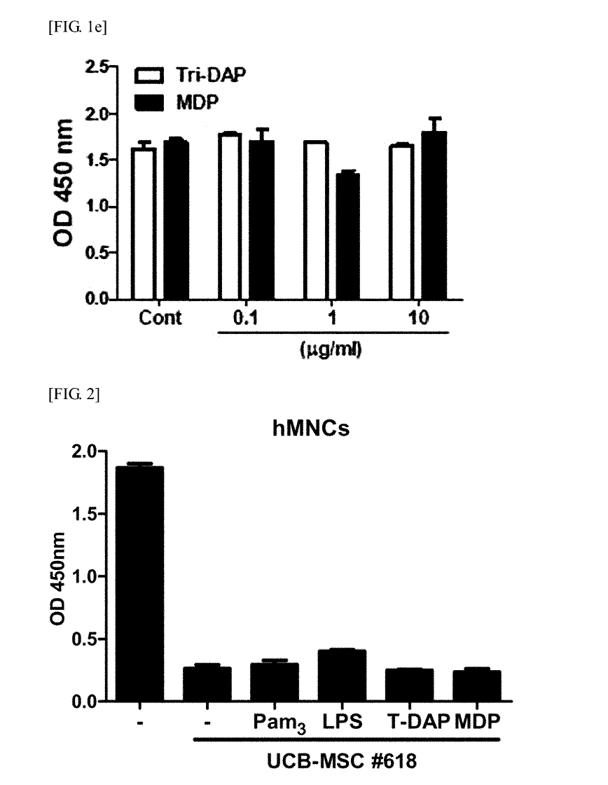

As shown in FIG. 2, hUCB-MSCs (#618) drastically inhibited the proliferation of human MNCs under direct cell to cell interaction. However, TLR (Pam3CSK4 and LPS) agonists and NLR agonists (Tri-DAP and MDP) did not affect the suppressive activity of hUCB-MSCs against MNC proliferation under the same condition (FIG. 2).

### EXAMPLE 3

### Identification of the Enhanced Immunosuppressive Activity of hUCB-MSCs by MDP Through NOD2-Rip2 Dependent Pathway (1)

3-1: Determination of the Effect of Secretory Factor Generated from MDP-Treated hUCB-MSC on MNC Proliferation

Soluble factors are also known to mediate immunosuppression by MSC. So the present inventors examined whether secretory factors generated by UCB-MSCs have an effect on human MNC proliferation. Culture medium (CM) was prepared, and hUCB-MSCs were co-cultured with the agonist for 24 hours. After washing, the cells were cultured in fresh medium for additional 4 days. Then, a control group containing culture medium (CM) and sample of hUCB-MSCs treated with agonist (#618) were prepared, and MNCs were cultured in CM containing hUCB-MSCs for 3 days.

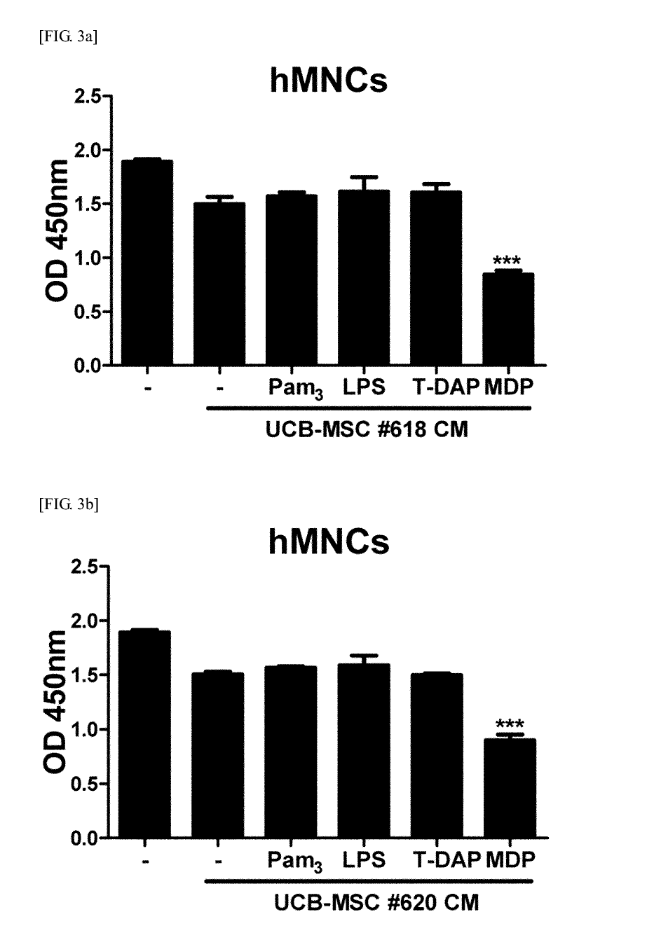

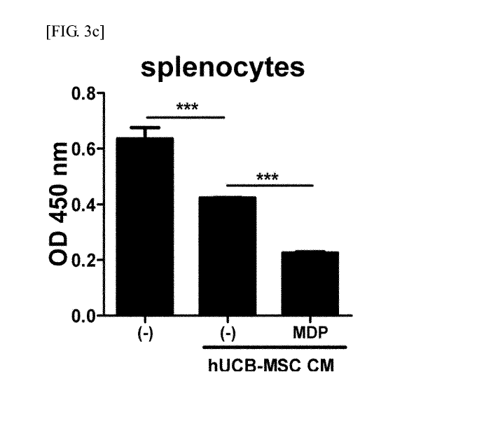

The experimental results demonstrated that MNC proliferation was slightly inhibited in the control group containing hUCB-MSC culture medium (UCM) (FIG. 3a). Surprisingly, when the MNCs were cultured in UCM that was pre-treated with MDP (MDP-UCM), MNC proliferation was inhibited at greater level, but this effect was absent when other agonists were used to treat UCM (Pam3CSK4, LPS, Tri-DAP) (FIG. 3a). The similar results were observed when UCM prepared from other sample of hUCB-MSCs (#620) was used (FIG. 3b). Furthermore, proliferation of xenogeneic mouse splenocytes was also inhibited when they were cultured in the presence of UCM, and this inhibitory effect was enhanced by MDP stimulation (FIG. 3c). These results suggest that secretory factors from MDP-treated hUCB-MSCs play an important role in immunosuppression of MNC.

3-2: Identification of the Enhanced Immunosuppressive Activity of hUCB-MSC by MDP Treatment

To determine whether MDP enhances the immunosuppressive activity of hUCB-MSC, the following experiment was performed. A control group containing culture medium (CM) of hUCB-MSC was prepared and the agonist-treated hUCB-MSCs were collected on the 5th day of culturing. Then, MNC was co-cultured in CM of hUCB-MSC for 3 days more, and after culturing, the level of MNC proliferation was monitored.

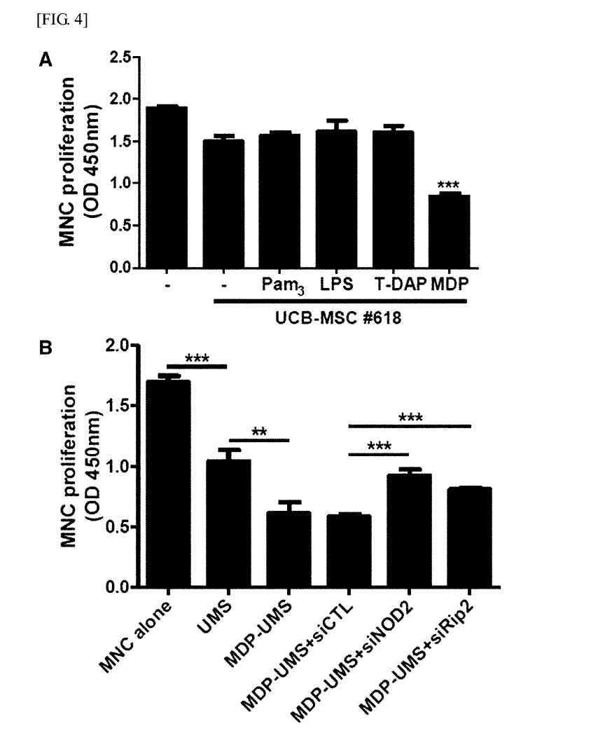

As shown in FIG. 4A, MNC proliferation was significantly inhibited in the supernatant of mesenchymal stem cells that were treated with the agonist MDP (UCB-MSC #618+MDP) compared to the untreated UCB-MSC supernatant (UCB-MSC #618).

On the other hand, the rate of MNC inhibition was similar in between the supernatants of mesenchymal stem cells that were treated with other agonists (LPS, etc.) (UCB-MSC #618+Pam3; UCB-MSC #618+LPS; UCB-MSC #618+T-DAP) and the untreated UCB-MSC supernatant (UCB-MSC #618). That is, compared to the untreated negative control group, the proliferation of MNC group was only slightly inhibited when treated with other agonist.

These results suggest that MNC proliferation is remarkably inhibited by soluble factors secreted from MDP-treated mesenchymal stem cells.

3-3: Investigation of the Correlation Among NOD2, Rip2 and MDP in MNC Inhibition by Using siRNA of NOD2 and Rip2

Additionally, to investigate the correlation among NOD2, Rip2 and MDP in MNC inhibition, the following experiment was conducted using siRNAs of NOD2 and Rip2 and a control group (siCTL). When the cell density reached 60%, siRNAs were transfected into the cells. The siRIPK2 (M-003602-02) which is the siRNA of receptor-interacting serine-threonine kinase 2 (RIPK2 or receptor interacting kinase protein 2 (Rip2) which is the adaptor of NOD1 and NOD2 and the type of kinase called RICK or CARDIAK (Bertin et al, 1999; Inohara et al, 1999; Ogura et al, 2001b)), siNOD2 (J-011388-07) which is the siRNA of NOD2, and a non-targeting control (siControl #1, D-001810-01) were purchased from Dharmacon (Chicago, Ill., USA). DharmaFECT1 (Dharmacon) was used as a transfection reagent, and siRNA was transfected at a concentration of 100 nmol/L. About 48 hours later, the medium was replaced with the fresh one, and the cells were treated with 10 μg/mL of MDP (NOD2 agonist) for 24 hours, except for a negative control (culture medium of MNC only) and a positive control (medium added with UCB-MSC supernatant (UMS) without agonist).

That is, a medium where MNC was cultured alone (i), and a medium added with UCB-MSC supernatant (UMS) without agonist (ii) were prepared as control groups, and a medium treated with MDP and UMS (iii), a medium treated with MDP, UMS, and control siRNA (siCTL) (iv), a medium treated with MDP, UMS and siNOD2 (v), and a medium treated with MDP, UMS, and siRip2 (vi) were prepared. Thereafter, MNC proliferation was measured by optical density at the wavelength of 450 nm.

As shown in FIG. 4B, the rate of MNC proliferation inhibition was similar in medium (iv) and medium (iii), whereas the rate of MNC proliferation inhibition in media (v) and (vi) was similar to that of medium (ii). In other words, the siRNAs of NOD2 and Rip2 could counteract the effect of MDP in enhancing the inhibition of MNC proliferation, but the control siRNA did not show the above effect. These results indicate that NOD2 and Rip2 positively regulate MDP-induced immune responses. Therefore, it is suggested that NOD2 and Rip2 are required for MDP-regulated-UCB-MNC inhibition.

### EXAMPLE 4

### Identification of the Enhanced Immunosuppressive Activity of hUCB-MSCs by MDP Through NOD2-Rip2 Dependent Pathway (2)

In order to verify the experimental results in Example 3, the mesenchymal stem cell line #620 obtained from Example 1 was used to perform the experiment following the same method described in Examples 3-2 and 3-3.

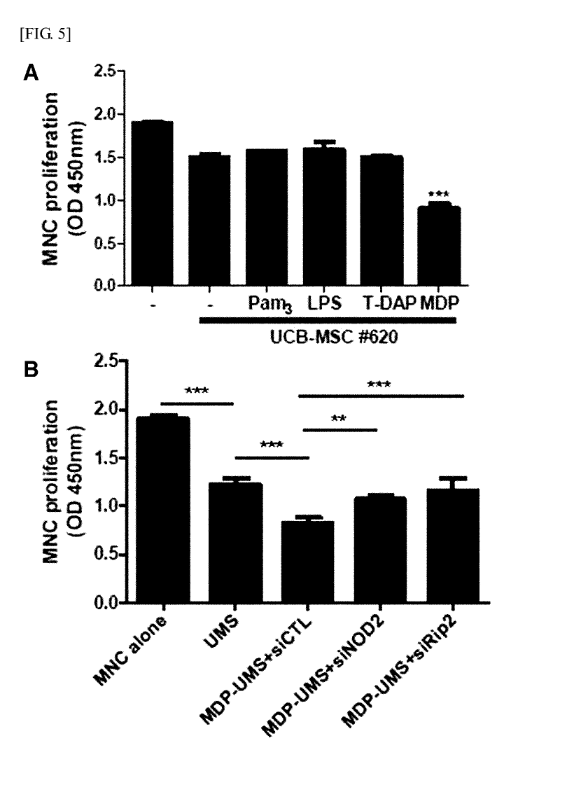

As shown in FIG. 5A, in the agonist MDP-treated mesenchymal stem cell supernatant (UCB-MSC #618+MDP) MNC proliferation was remarkably inhibited as compared to the UCB-MSC supernatant cultured with other receptor agonists or cultured without any agonist (UCB-MSC #620), which are the similar results as observed in Example 3-1. As shown in FIG. 5B, the effect of MDP in enhancing MNC inhibition was counteracted by siRNAs of NOD2 and Rip2, but not by the control siRNA, which is also similar to the results of Example 3-3. These results indicate that MDP enhances suppressive activity of mesenchymal stem cells against MNC proliferation via NOD2-Rip2-dependent pathway.

Together with the results of Example 3, the above results suggest that the MDP-treated stem cells of the present invention and the culture thereof demonstrate strong immunosuppressive effects, and thus can be used as an immunoregulatory composition for the treatment of autoimmune diseases such as rheumatoid arthritis and Crohn's disease or immune disorders such as atopic dermatitis.

### EXAMPLE 5

### Analysis of the Correlation Between MDP-Induced PGE2 Production and MNC Inhibition by UMS (UCB-MSC Supernatant; UMS)

5-1: Increase in PGE2 Secretion from MSC by MDP Stimulation

The hUCB-MSCs (2×104 cells/well) were cultured in MSC medium supplemented with 2% FBS in a 96-well plate. After 24 hrs of culturing, the cells were treated with 1 μg/mL Pam3CSK4 (TLR2 agonist), 1 μg/mL LPS (TLR4 agonist), 10 μg/mL Tri-DAP (NOD1 agonist) or 10 μg/mL MDP (NOD2 agonist) and cultured for additional 24 hours, then the culture supernatant of each sample was collected. After centrifugation, the culture supernatants were filtered through a 0.2 μm filter. Then, PGE2 concentration was measured using an ELISA kit (R&D Systems, Minneapolis, Minn., USA) following the manufacture's protocol.

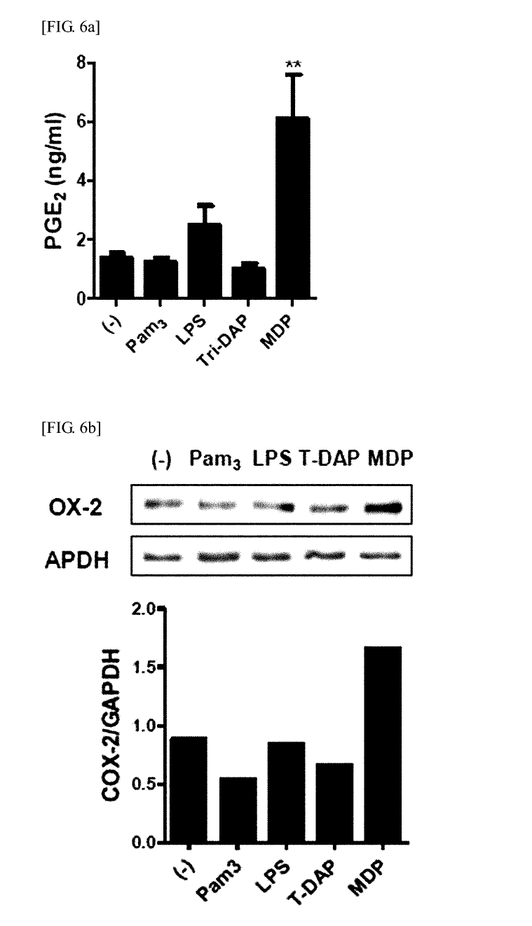

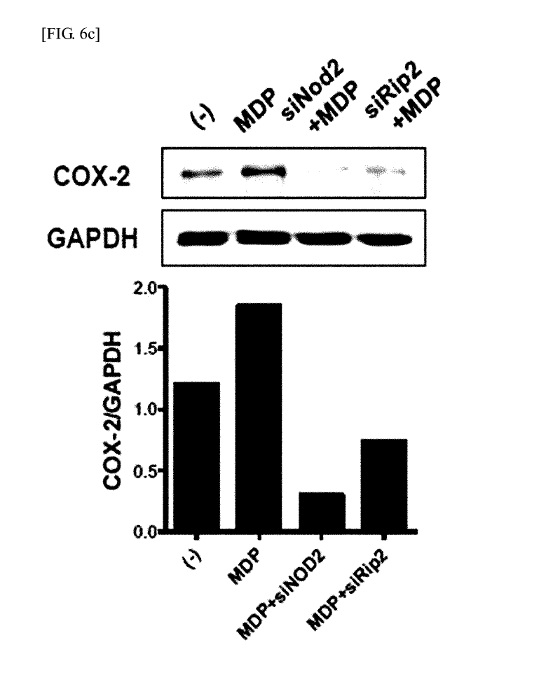

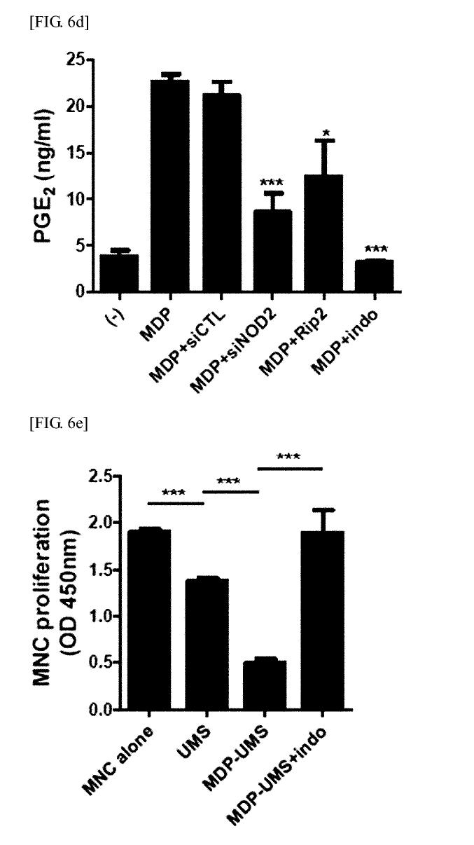

As shown in FIG. 6a, treatment of hUCB-MSCs with the NOD2 agonist, MDP, significantly enhanced PGE2 secretion, as compared to those treated with the agonists of other receptors.

5-2: Analysis of the Correlation Between COX-2 Expression and MDP Treatment

Cells were treated with 1 μg/mL Pam3CSK4 (TLR2 agonist), 1 μg/mL LPS (TLR4 agonist), 10 μg/mL Tri-DAP (NOD1 agonist) or 10 μg/mL MDP (NOD2 agonist) and cultured for 24 hours. Then, the collected cells were lysed using 1% Nonidet-P40 buffer containing 2 mM dithiothreitol and protease cocktail (Roche, US). The cell lysates were resolved by 12% SDS-PAGE, and transferred onto a nitrocellulose membrane. Then, immunostaining was performed using primary antibodies (COX-2, GAPDH (Santa Cruz biotechnology, Santa Cruz, Calif., USA)). Thereafter, immunostaining was performed using secondary antibodies, and proteins were detected using an enhanced chemiluminescence (ECL) reagent (Intron Biotechnology).

As shown in FIG. 6b, hUCB-MSCs treated with the NOD2 agonist, MDP, showed an enhanced COX-2 expression, as compared to those treated with the agonists of other receptors.

5-3: Analysis of the Correlation Between COX-2 Expression and the Activity of NOD2 and Rip2 Stimulated by MDP Treatment

To investigate a correlation between COX-2 expression and the function of NOD2 and Rip2, cells were first treated with MDP and further treated with siNOD2 and siRip2. Then the COX-2 expression level was examined. Protein expression level was measured by using the method described in Example 5-2, and the method for siRNA treatment was the same as described in Example 3-3.

As shown in FIG. 6c, COX-2 expression level was reduced by inhibition of NOD2 and Rip2. This result suggests that COX-2 expression depends on the activity of NOD2 and Rip2.

5-4: Investigation of the Effects of NOD2, Rip2 and COX-2 on PGE2 Expression

To determine the sustainment time of the effects of MDP treatment, the amount of PEG2 produced was monitored after 1 day-long treatment of hUCB-MSCs with MDP. In this experiment, MDP was removed after 1 day of culturing by removing the culture medium and washing the cells with phosphate buffered saline (PBS) 5 times. Then the amount of PEG2 produced was measured. Meanwhile, cells were treated with MDP by the same method described in Example 5-1, and PGE2 concentration was measured. Also, in order to determine the effects of NOD2, Rip2 and COX-2 on PGE2 expression, the cells were treated with each of the control group of Example 3-3 i.e. siRNA (siCTL), siNOD2, siRip2 and COX-2 inhibitor called indomethacin (Sigma (St. Louis, Mo., USA).

As shown in FIG. 6d, even when MDP was removed after 1 day of treatment, PGE2 production level in the MDP-stimulated hUCB-MSCs was greater than that in the control group on the 5th day of culturing. In addition, MDP-enhanced PGE2 production was inhibited by transfection with the NOD2 and Rip2 siRNAs or by inhibition of COX-2 by adding indomethacin. These results indicate that NOD2, Rip2 and COX-2 take an important role in MDP-induced PGE2 production in hUCB-MSCs.

5-5: Investigation of the Role of PGE2 in Inhibition of MNC by hUCB-MSC

In order to investigate the role of PGE2 in inhibition of MNC by hUCB-MSC, MNC proliferation was examined when treated with the MSC supernatant, MDP-treated MSC supernatant, and MDP and indomethacin (COX-2 inhibitor)-treated MSC supernatant. The experiment was performed by the same method described in Example 3.

As shown in FIG. 6e, cell treatment with the MDP-treated MSC culture medium significantly inhibited MNC proliferation, but co-treatment with indomethacin (Indo) showed MNC proliferation rate similar to that of the negative control group. These results indicate that PGE2 takes an important role in the inhibition of MNC by hUCB-MSC, which is consistent with the result of Example 5-4.

These results suggest that the MDP-treated stem cells of the present invention and the culture thereof produce PGE2, which can be used as an immunoregulatory composition. As aforementioned, since PGE2 is known to inhibit secretion of cytokines such as interleukin-1 beta and TNF alpha, the MDP-treated stem cells of the present invention and the culture thereof can also be used as an anti-inflammatory composition.

5-6: Investigation of the Effect of NOD2 and COX-2 on MDP-Induced Production of Anti-Inflammatory Cytokine Interleukin-10 (IL-10)

In order to determine the production level of IL-10, hUCB-MSCs (1×105 cells/well) were cultured in MSC medium supplemented with 2% FBS in a 24 well plate. About 24 hours later, the cells were treated with siNOD2 or indomethacin (Indo), and further cultured for 24 hours. Then the cells were washed five times, and fresh RPMI was added. After 5 days of culturing, UCB-MSC supernatant (UMS) was obtained. MNCs (1×106/well) were cultured with UMS and ConA (Sigma (St. Louis Mo., USA)). After 3 days of culturing, cell supernatant was collected, centrifuged, and filtered through a 0.2 μm filter. Then, IL-10 concentration was measured using an ELISA kit (R&D Systems, Minneapolis, Minn., USA).

As shown in FIG. 6f, production of the anti-inflammatory cytokine, IL-10, was remarkably increased by MDP treatment. However, IL-10 production was suppressed by inhibition of NOD2 or indomethacin treatment. These results suggest that MDP treatment increases the production of the anti-inflammatory cytokine IL-10 by acting on NOD2-Rip2 pathway, in which is the same pathway involved in production of PGE2.

In other words, the MDP-treated stem cells of the present invention produce anti-inflammatory cytokine IL-10 at high yield, and thus the MDP-treated stem cells or the culture thereof can be used as an anti-inflammatory composition, in particular, for the treatment of arthritis or the like.

### EXAMPLE 6

### Analysis of the Correlation Between MNC Inhibition and MDP-Induced Production of PGE2 and TGF-β in hUCB-MSCs

6-1: Investigation of the Effect of MDP on the Production of PGE2 and TGF-β1 in MSC and the COX-2 Expression

Soluble factors such as hepatocyte growth factor, TGF-β, indoleamine 2,3 dioxygenase-1 (IDO-1), nitric oxide (NO), and prostaglandin E2 (PGE2) are the strong candidates for regulating immunosuppression by MSC. In order to determine whether TLR and NLR agonists induce the production of soluble factors including NO, PGE2 and TGF-β1 in hUCB-MSCs, the cells were cultured with Pam3CSK4, LPS, Tri-DAP, and MDP for 24 hours, and culture supernatants were collected. Secretion of the soluble factors was monitored by the method described in Examples 5-1 and 5-2.

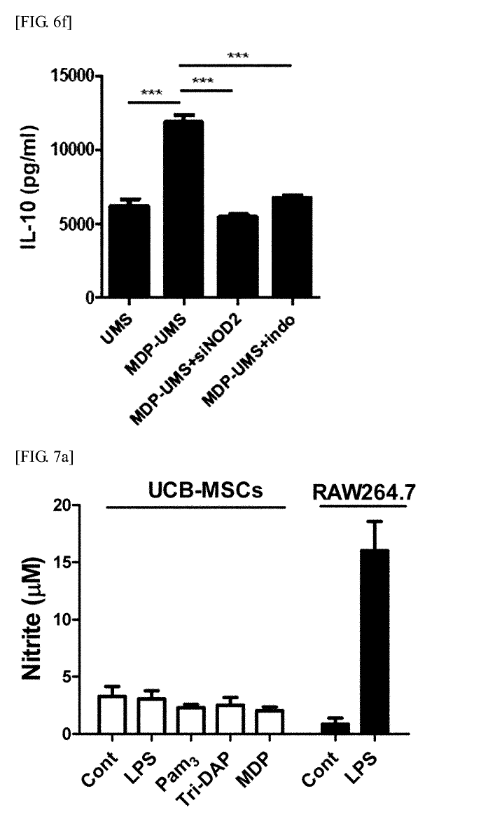

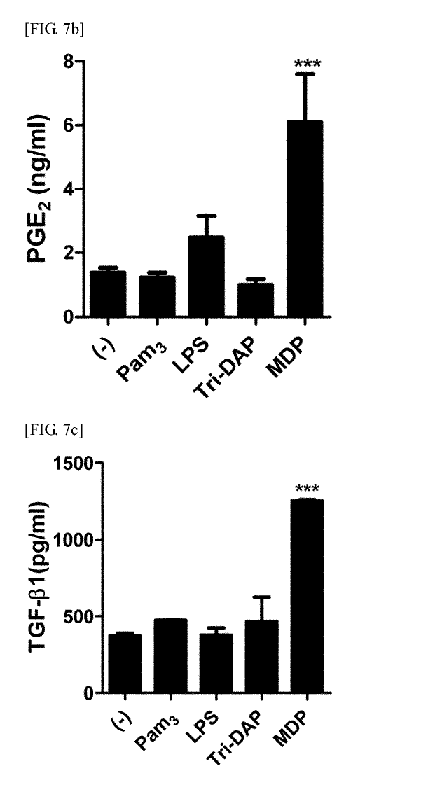

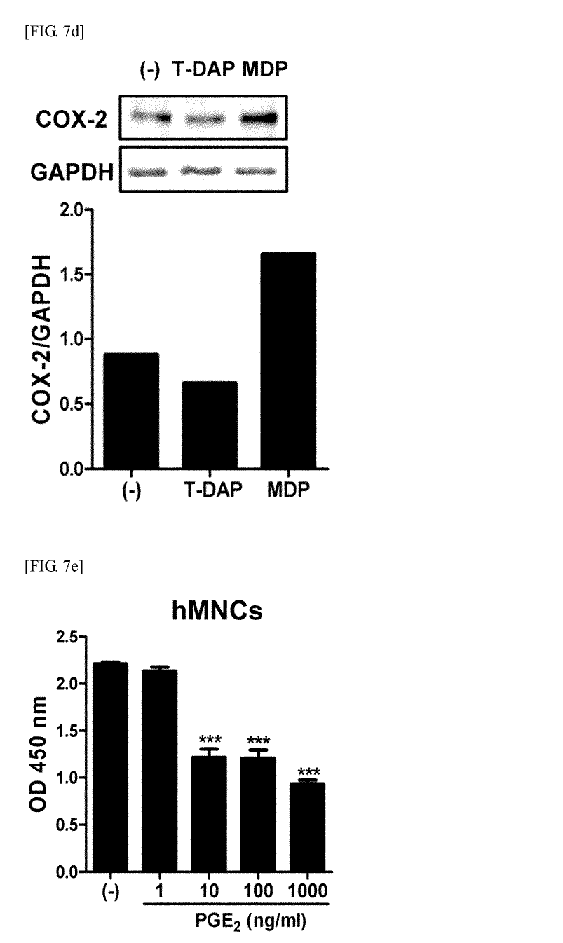

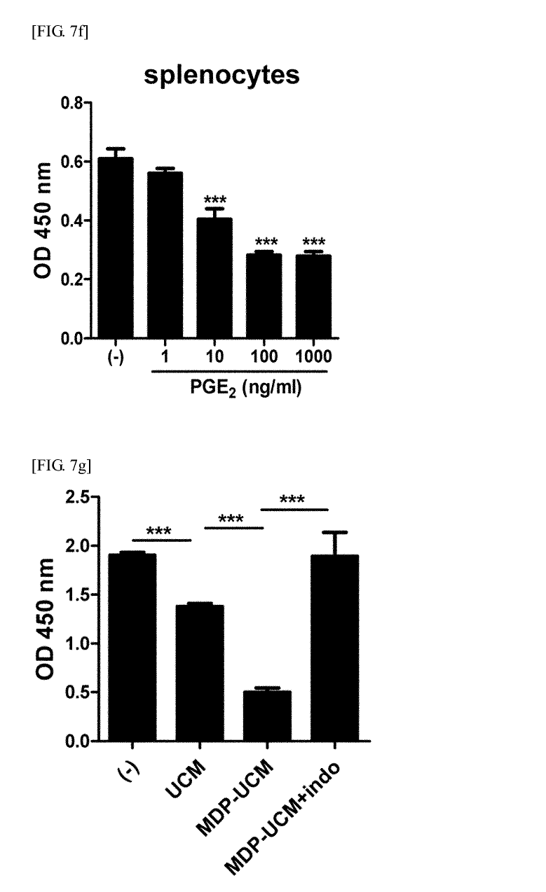

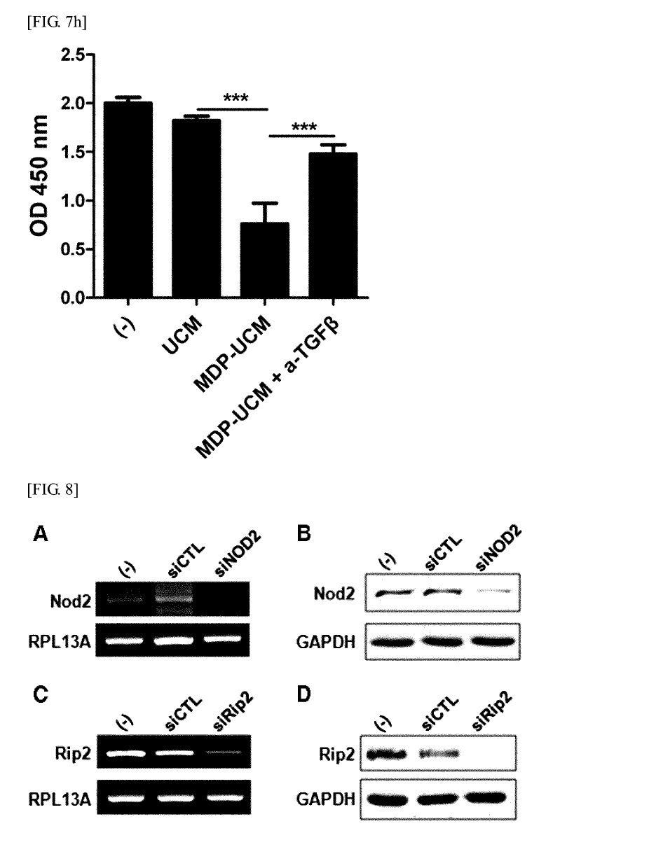

The results demonstrate that single treatment with TLR and NOD agonists did not induce the production of NO in hUCB-MSCs, even though LPS induced the production of NO in macrophages (FIG. 7a). Interestingly, the production of PGE2 and TGF-β1 was enhanced only by addition of MDP in hUCB-MSCs but not by other agonists (FIGS. 7b and 7c). Moreover, expression of COX-2, the PGE2-producing enzyme, was increased in hUCB-MSCs after 24 hours of MDP treatment (FIG. 7d).

6-2: Investigation of the Effect of PGE2 and TGF-β on MNC Proliferation

In order to investigate the effect of PGE2 on mononuclear cell (MNC) proliferation, hMNC and mouse splenocytes were cultured with various concentrations of PGE2. The experiment was performed by the same method described in Example 5-5.

The results showed that proliferation of hMNC and mouse splenocytes was remarkably inhibited when cultured with PGE2 at a concentration of 10 ng/mL or higher (FIGS. 7e and 7f).

Next, the present inventors examined whether hMNC inhibition by MDP-pretreated UCM is attributed to PGE2 and TGF-β1. The experiment was performed by the same method described in Example 5-5.

The results showed that the inhibitory effect of MDP-UCM on hMNC proliferation was counteracted by a COX inhibitor indomethacin (FIG. 7g). Furthermore, when co-treated with TGF-β1 neutralizing antibody, MDP-UCM did not inhibit hMNC proliferation (FIG. 7h). These results suggest that MDP induces the production of PGE2 and TGF-β1 in hUCB-MSCs, which mediates the immunosuppressive activity of hUCB-MSCs.

### EXAMPLE 7

### Analysis of the Correlation Between the MDP-Induced COX-2 Expression and PGE2 and TGF-β1 Production in hUCB-MSCs and the Activity of NOD2 and Rip2

NOD2 and Rip2 are the important factors in MDP-induced immune responses. Therefore, the present inventors examined whether NOD2 and Rip2 are required in the MDP-induced COX-2 expression and production of PGE2 and TGF-β1 in hUCB-MSCs. The experiment was performed by the same method described in Example 5-4.

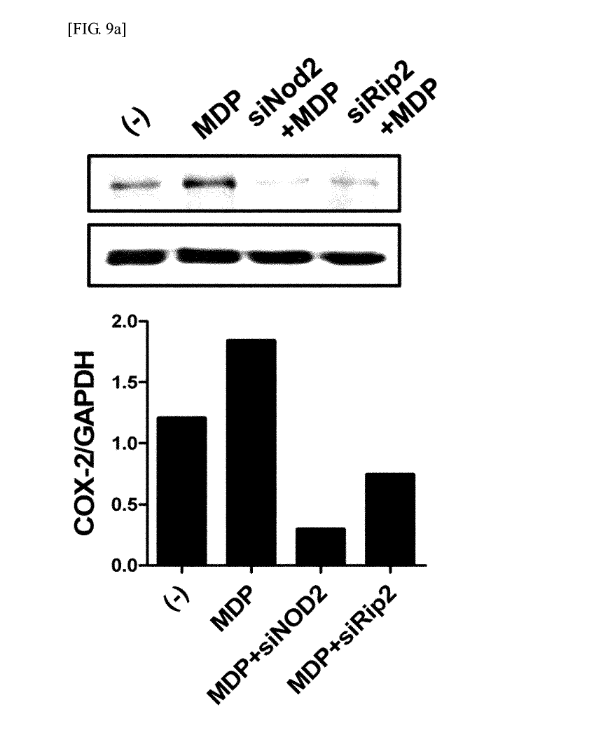

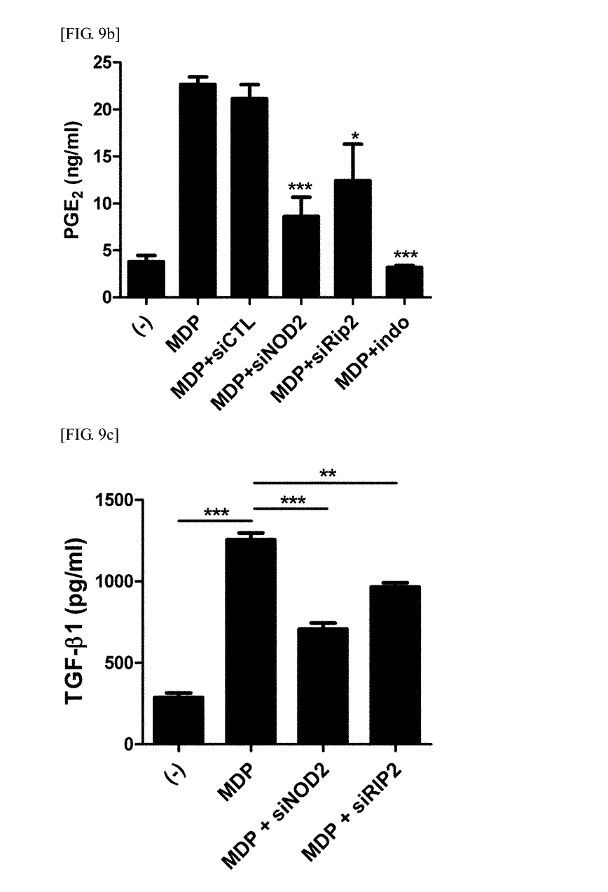

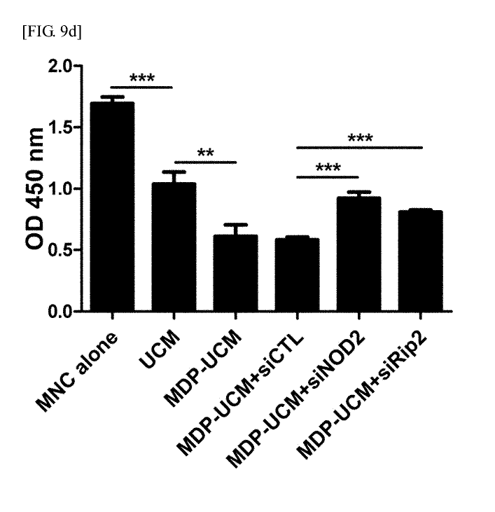

The results demonstrated that the gene and protein expression of both NOD2 and Rip2 are remarkably suppressed by siRNAs in hUCB-MSCs (FIG. 8). Down-regulation of NOD2 and Rip2 by siRNA further suppressed MDP-induced COX-2 expression in hUCB-MSCs (FIG. 9a). The siRNA treatment of NOD2 and Rip2 reduced PGE2 and TGF-β1 productions in MDP-UCM, as compared to the control siRNA (FIGS. 9b and 9c). Furthermore, MLR experiment showed that down-regulation of NOD2 and Rip2 restored the increased inhibitory effect of MDP-UCM against hMNC proliferation (FIG. 9d). These results suggest that MDP induces the increased production of PGE2 and TGF-β1 in hUCB-MSCs via NOD2-Rip2 pathway, indicating enhancement of immunosuppressive ability of UCM-MSC.

### EXAMPLE 8

### Analysis of the Effect of MDP-UCM on IL-10 Production and Regulatory T Cell Population in hMNCs

8-1: Investigation of the Effect of MDP-UCM on IL-10 Production in hMNCs

PGE2 produced in bone marrow stromal cells is known to take an important role in IL-10 production by host macrophages (Nemeth, K., A. et al., Bone marrow stromal cells attenuate sepsis via prostaglandin E(2)-dependent reprogramming of host macrophages to increase their interleukin-10 production. Nat Med 15:42-49, 2009). MDP induces PGE2 production in hUCB-MSCs, and thus the present inventors examined whether IL-10 production in hMNCs is increased in the presence of MDP-UCM. The experiment was performed by the same method described in Example 5-6.

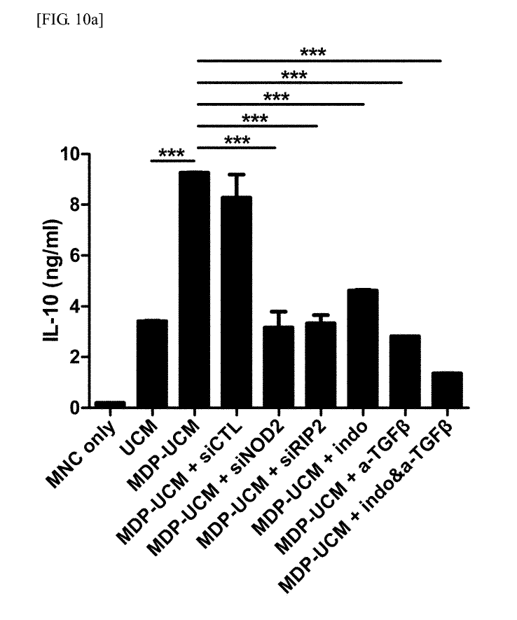

The results demonstrated that UCB-MSC alone did not produce IL-10, regardless of MDP stimulation (data not shown). Although hMNC alone did not produce IL-10, IL-10 production was up-regulated in the presence of UCM (FIG. 10a). Moreover, IL-10 production by hMNC was more increased in the presence of MDP-UCM than in the presence of the untreated UCM (FIG. 10a). However, the increased IL-10 production in hMNC by MDP-UCM was reversed by single treatment with indomethacin or TGF-β1 neutralizing antibody, and the above effect was accelerated by treating the cell with a combination of indomethacin and TGF-β1 neutralizing antibody (FIG. 10a). When NOD2 activity was suppressed by siRNA in hUCB-MSCs, IL-10 production in hMNC was not increased even in the presence of MDP-UCM, compared to non-treated UCM (FIG. 10a).

8-2: Investigation of the Effect of MDP-UCM on T Cell Population in hMNCs

Next, the present inventors investigated the effect of UCM on differentiation of hMNC into regulatory T cells (Treg). For this, hMNCs were cultured with UCM for 5 days, and co-expression of CD4, CD25, and Foxp3 in hMNCs was determined by Flow cytometry.

The results demonstrated that Treg population in hMNCs was increased by 50% or more in the presence of UCM, and the increase in Treg population was higher in hMNCs cultured with MDP-UCM than in those cultured with untreated UCM (FIG. 10b). Similarly, the increase in Treg population by MDP-UCM was reversed by treatment with indomethacin and TGF-β1 neutralizing antibody or by NOD2 inhibition with siRNA (FIG. 10b). These results indicate that MDP-induced production of PGE2 and TGF-β1 in UCM is important in IL-10 production by hMNC and differentiation of hMNC into Treg.

### EXAMPLE 9

### Determination of NOD2 Expression in Other Types of Stem Cells

In order to determine whether immune and inflammatory responses can be regulated by the interaction between NOD2 receptor and agonist for other types of stem cells by addition of Nucleotide-binding Oligomerization Domain protein 2 (NOD2) agonist, NOD2 expressions in adipose tissue-derived stem cells (AD-MSC) and amniotic epithelial stem cells (AEC) were examined using a human monocytic leukemia cell line, i.e. THP-1 as a positive control group. RT-PCR was performed under the same condition described in Example 2. Human amniotic epithelial stem cells were obtained after delivery, with the written informed consent of the patient approved by the Guro Hospital (the Seoul National University IRB (IRB No. 0611/001-002), and amniotic epithelial stem cells were isolated from the obtained amnion tissue. The adipose tissue-derived stem cells were obtained, with the written informed consent of the patient approved by the Boramae Hospital (SNU IRB #0600/001-001), and the adipose tissue-derived stem cells were isolated and cultured.

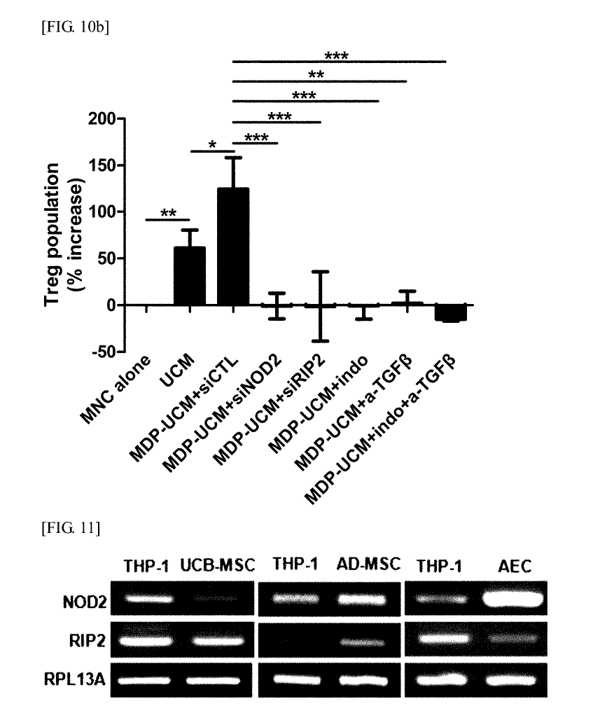

As shown in FIG. 11, NOD2 expression was higher in adipose tissue-derived stem cells and amniotic epithelial stem cells than in the positive control i.e., THP-1 cells. In other words, NOD2 receptor expression was observed in other types of stem cells as well as in mesenchymal stem cells, suggesting that other types of stem cells can be also used to regulate immune and inflammatory responses via NOD2 receptor-agonist interaction.

### EXAMPLE 10

### Investigation of the Therapeutic Effects of NOD2-Expressing Stem Cells Using Colitis Animal Model

10-1: Investigation of the Therapeutic Effects of NOD2-Expressing Stem Cells Using Colitis Animal Model

The present inventors examined whether hUCB-MSC treatment is effective for treatment of colitis mouse and whether MDP stimulation promotes protective effects of hUCB-MSC on DSS-induced colitis. For administration of hUCB-MSC, cells were cultured in the presence or absence of MDP for 24 hours, and then washed with PBS to remove MDP. To be specific, in order to investigate the therapeutic effects of MDP-treated stem cells in colitis animal models, colitis was induced in mice (Central Lab. Animal Inc., C57BL/6N) by treatment with 3% DSS (dextran sulfate sodium). After 2 days of treatment, MDP-treated hUCB-MSC, non-treated hUCB-MSC, and NOD2-suppressed hUCB-MSC by treatment with siRNA were intraperitoneally injected. At this time, 3% DSS was administered in drinking water for 7 days.

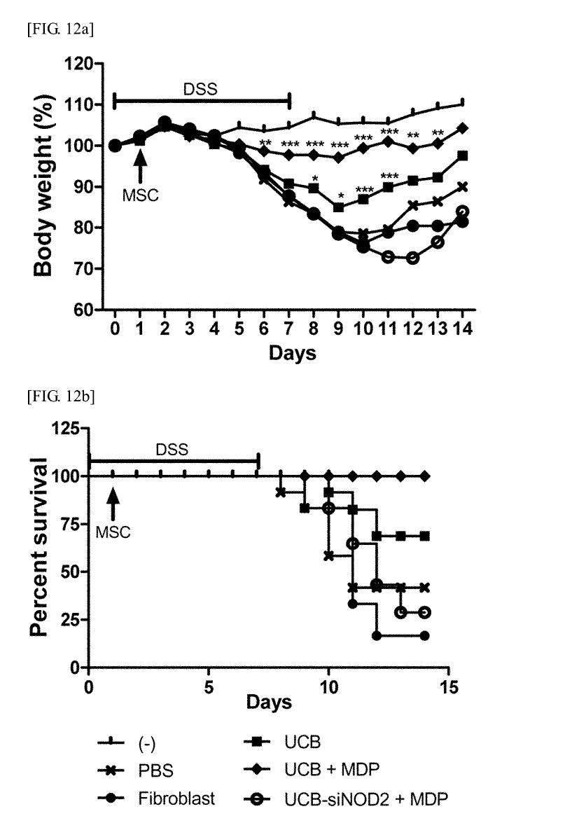

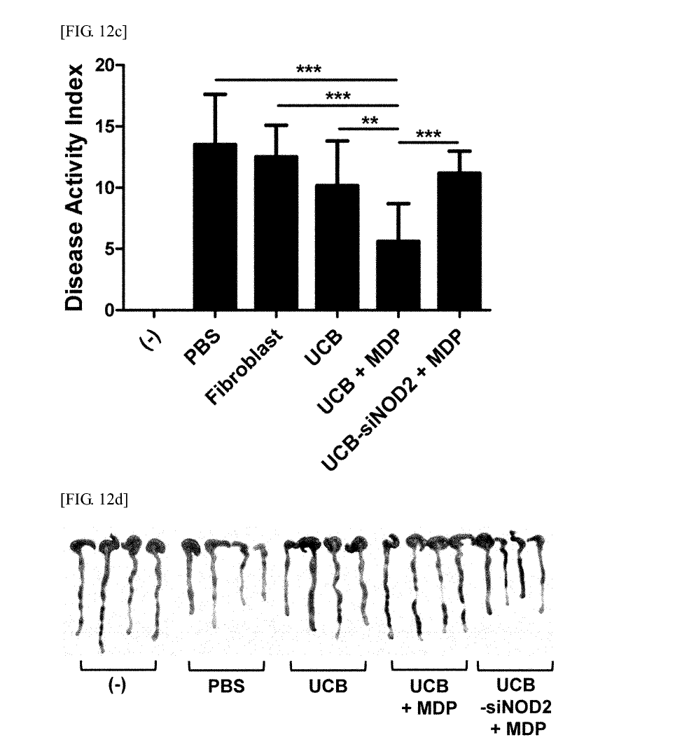

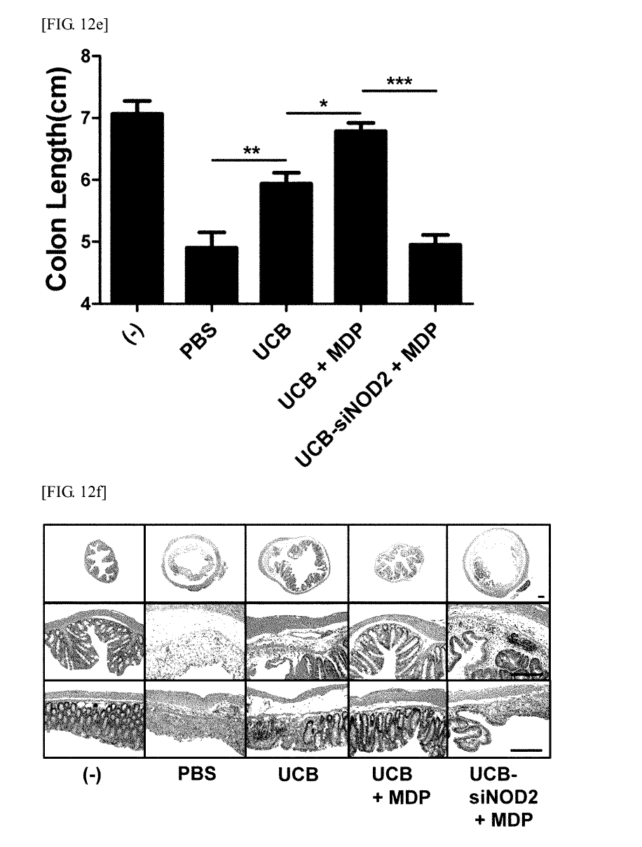

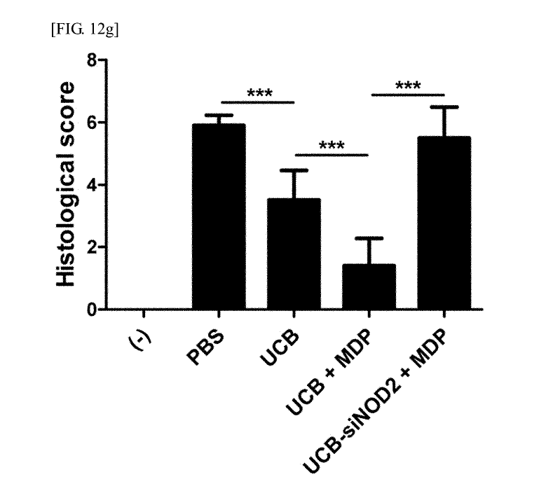

The results demonstrated that intraperitoneal injection of non-treated hUCB-MSC alleviated the body weight reduction and also improved survival rate of DSS-induced colitis mouse, as compared to PBS- or fibroblast-treated mouse (FIGS. 12a and 12b). Moreover, injection of MDP-stimulated hUCB-MSC (MDP-MSC) led to recovery of the body weight to 90% of the control group which was free of colitis, and none of the mice died from colitis (FIGS. 12a and 12[[B]]b). Disease activity index was slightly reduced by injection of hUCB-MSC, but it was significantly different from that of PBS- or fibroblast-treated group. On the other hand, injection of MDP-MSC significantly reduced disease activity index (FIG. 12c). When NOD2 was down-regulated by siRNA, MDP-MSC did not alleviate body weight reduction, or increase survival rate and disease activity index.

Mice were sacrificed on the 14th day to measure their colon length, and histopathological analysis was performed. The results of gross examination showed that the reduced colon length by inflammation was slightly alleviated by treatment with hUCB-MSC, which was enhanced by addition of MDP (FIGS. 12d and 12e). The results of histopathological study showed that colonic mucosal erosions, severe edema lesions, and inflammatory cell infiltration of the lamina propria and submucosal layer were observed in DSS-treated mice (FIG. 12f). The mucosal erosions of the submucosal layer and edema confined to a part of the colon were observed in hUCB-MSC-treated mice (FIG. 12f). However, the pathologic damages in the colons were completely treated and pathological scores were remarkably reduced by treatment with MDP-MSC (FIGS. 12f and 12g). As expected, injection of siNOD2-treated hUCB-MSC neither relieved pathological severity nor reduced pathological scores in DSS-induced colitis (FIGS. 12f and 12g). These results suggest that MDP enhances the protective effect of hUCB-MSC on colitis, indicating a critical role of NOD2.

10-2: Investigation of the Effect of MDP on Inflammatory Cytokine Production and T Cell Populations in Colitis Animal Model

Furthermore, the present inventors investigated the effect of MDP on the regulation of hUCB-MSC-induced inflammatory cytokine production in colitis mouse.

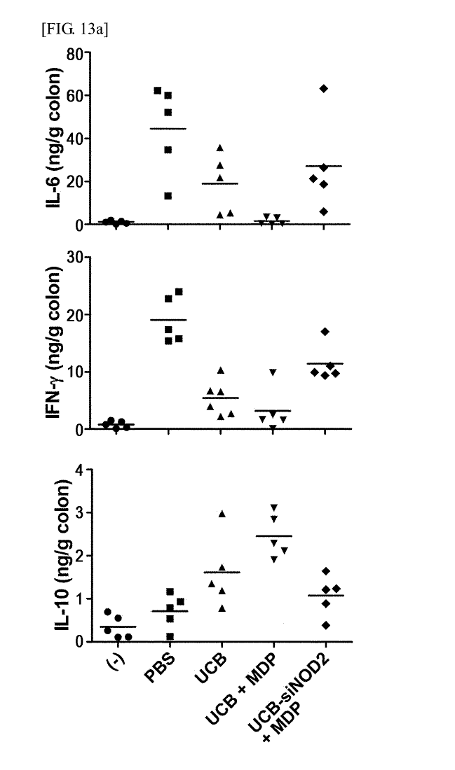

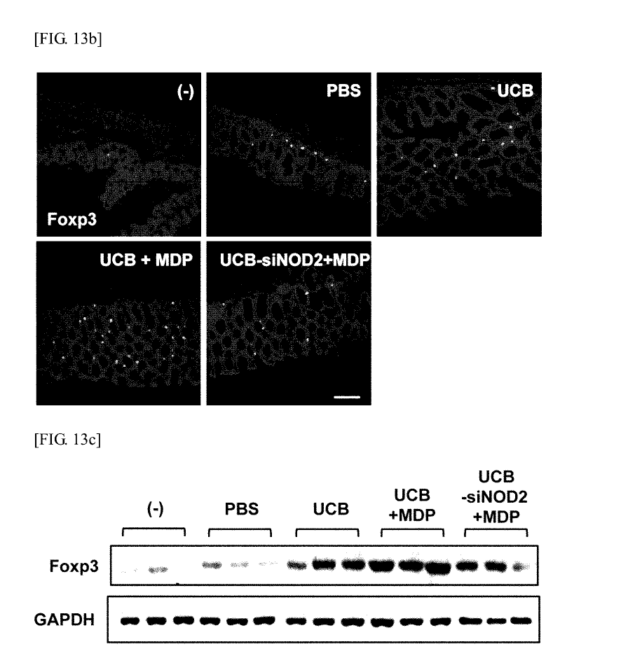

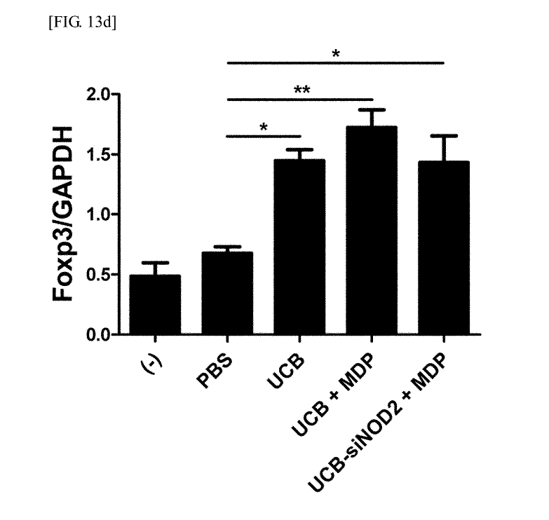

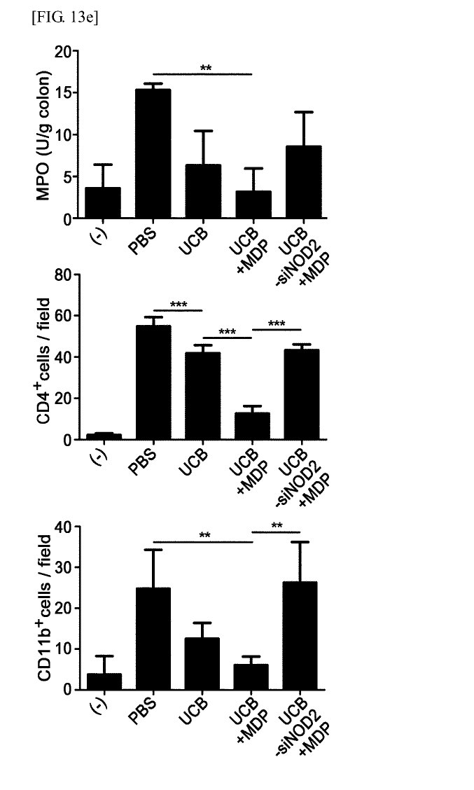

The results showed that hUCB-MSCs reduced IL-6 and IFN-γ productions and increased IL-10 production in DSS-treated mouse colons (FIG. 13a). Moreover, MDP-MSC blocked IL-6 and IFN-γ productions almost completely and promoted IL-10 production in DSS-treated mouse colons, but this effect was counteracted by NOD2 down-regulation (FIG. 13a).

In order to determine whether hUCB-MSCs have an effect on Treg populations in the mouse colon, Foxp3+ cell infiltration into colon was monitored under a fluorescence microscope.

The results demonstrated that localization of Fox3p+ cells in the colon was higher in hUCB-MSC-treated colitis mice than in PBS-treated colitis mice (FIG. 13b). In addition, localization of Fox3p+ cells in the colon was increased further by treatment with MDP-MSC, which was counteracted by NOD2 inhibition in hUCB-MSC (FIG. 13b). The Foxp3 expression in the colon was quantified by Western blotting. Foxp3 protein expression was higher in hUCB-MSC-treated mice than in PBS-treated colitis mice (FIGS. 13c and 13d). Similarly, Foxp3 level in the colon was increased further by treatment with MDP-MSC, which was also counteracted by NOD2 down-regulation (FIGS. 13c and 13d).

10-3: Analysis of Inflammatory Cell Infiltration in Colitis Animal Model

Lastly, the present inventors examined inflammatory cell infiltration into mouse colon. The activity of myeloperoxidase (MPO) was monitored to determine neutrophilic infiltration.

The results demonstrated that hUCB-MSC suppressed MPO activity and CD4+ and CD11b+ cell infiltration into the DSS-treated mouse colon (FIG. 13e). Likewise, MDP-MSC suppressed MPO activity and CD4+ and CD11b+ cell infiltration to a greater extent, which was counteracted by NOD2 inhibition by siRNA (FIG. 13e).

These results support that hUCB-MSC induces anti-inflammation while suppressing pro-inflammatory response, which can be enhanced by MDP treatment in a NOD2-dependent manner.

Furthermore, these results suggest that the MDP-treated stem cells of the present invention or the culture thereof can be practically used for the treatment of inflammation in animal models having colitis.

### EXAMPLE 11

### Investigation of the Therapeutic Effect of MDP-Treated hUCB-MSC on Atopic Dermatitis-Induced Animal Model

11-1: Induction of Atopic Dermatitis and Treatment with hUCB-MSC Injection

One day before conducting the present experiment, the hair on the back of 8-week-old female NC/Nga mouse was shaved, and the remaining hair on the back skin was completely removed by applying a hair removal cream. Then the next day, 150 μl of 4% sodium dodecyl sulfate (SDS) aqueous solution was applied to the shaved back skin to remove fat from the skin. The skin was completely dried for approximately 3 to 4 hours, and then 100 mg of Dermatophagoides farina (Df) extract was applied evenly to the back and ears. Application of Df extract was performed twice a week for 3 weeks for a total of 6 times (application of SDS aqueous solution is essential for every application of Df extract) to induce atopic dermatitis. Then, 2×106 hUCB-MSCs prepared in Example 1 was suspended in 200 μl of phosphate buffered saline (PBS), and intravenously or subcutaneously injected to NC/Nga mouse once a week for 3 weeks during Df application and 1 week after induction of atopic dermatitis, i.e. a total of 4 times for 4 weeks. MDP-treated hUCB-MSCs were prepared by culturing hUCB-MSCs in a medium supplemented with 10 μl/mL of MDP for 24 hours, and then by washing the cells with PBS 5 times prior to injection.

11-2: Investigation of the Therapeutic Effect of MDP-Treated hUCB-MSC by Gross Examination of the Lesion

In order to investigate the therapeutic effect of the MDP-treated hUCB-MSCs on atopic dermatitis, an autopsy was conducted after 24 hours of the 4th injection of hUCB-MSCs, and severity of the lesion was evaluated by gross examination. The gross examination was conducted in accordance with dryness, excoriation, erythema, and edema giving a score of 0 to 3, and the total score was used for evaluation.

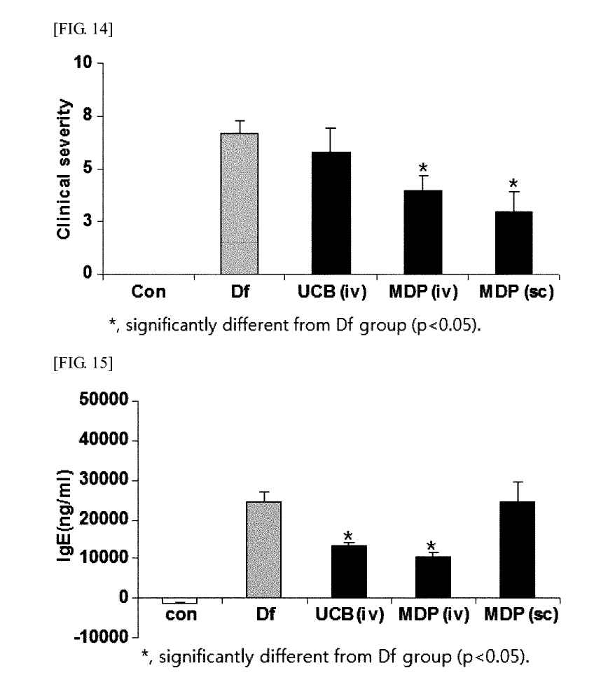

As shown in FIG. 14, intravenous injection of hUCB-MSC into atopic dermatitis-induced group induced a slight alleviation of lesions, not having a great difference from non-treated atopic dermatitis-induced group. In contrast, significant alleviation of lesions was observed in the groups that had intravenous or subcutaneous injection of MDP-treated hUCB-MSC.

These results suggest that the MDP-treated stem cells of the present invention or the culture thereof can be used for the treatment of autoimmune diseases such as atopic dermatitis.

11-3: Investigation of the Therapeutic Effect of MDP-Treated hUCB-MSC by Analysis of Serum IgE

In order to investigate the therapeutic effect of MDP-treated hUCB-MSCs on atopic dermatitis, an autopsy was conducted after 24 hours of the 4th injection of hUCB-MSCs, and IgE level in the serum collected from autopsy was measured using a commercial Opt EIA mouse set (BD Bioscience, Mississauga, Canada).

As shown in FIG. 15, a significant inhibition of IgE was observed in the groups that had intravenous injections of hUCB-MSC or MDP-treated hUCB-MSC, while a higher inhibition rate was observed in the group that had intravenous injection of MDP-treated hUCB-MSC. However, subcutaneous injection of MDP-treated hUCB-MSC did not induce a great difference from the atopic dermatitis-induced group.

11-4: Investigation of the Therapeutic Effect of MDP-Treated hUCB-MSC by Analysis of Serum IgG1

In order to investigate the therapeutic effect of MDP-treated hUCB-MSCs on atopic dermatitis, an autopsy was conducted after 24 hours of the 4th injection of hUCB-MSCs, and IgG1 level which is a representative index for a Th2 immune response of atopic dermatitis was measured in the serum collected from autopsy using an ELISA kit (Bethyl Laboratories Inc., Montgomery, Tex., USA).

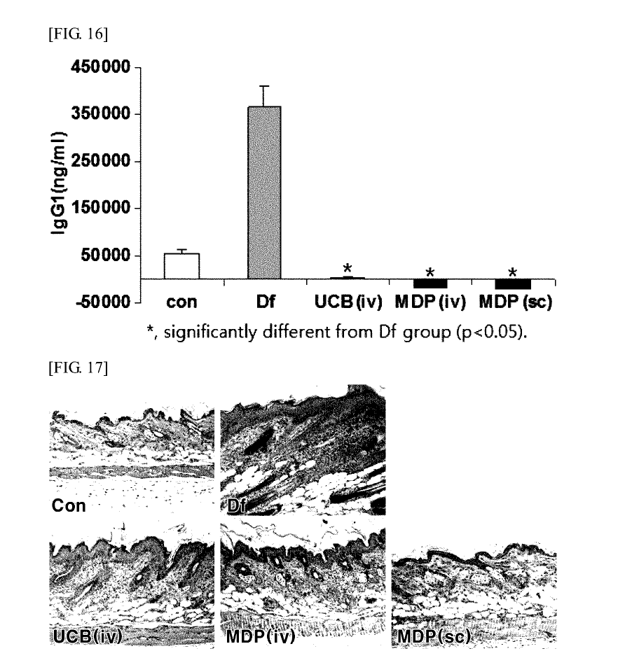

As shown in FIG. 16, a significant inhibition of IgG1 was observed in all of the hUCB-MSC-treated groups.

11-5: Investigation of the Therapeutic Effect of MDP-Treated hUCB-MSC by Histopathological Examination of Skin Tissue

In order to investigate the therapeutic effect of MDP-treated hUCB-MSCs on atopic dermatitis, an autopsy was conducted after 24 hours of the 4th injection of hUCB-MSCs, and the skin tissues were collected and fixed with a 10% neutral formalin solution. Then, the tissue slices were processed, paraffin-embedded, and cut into 3 to 4 μm sections. Then, hematoxylin-eosin (H&E) staining was performed for pathological study.

As shown in FIG. 17, epidermal hyperplasia and excessive infiltration of inflammatory cells were observed in the atopic dermatitis-induced group. Reductions in epidermal thickness and infiltration of inflammatory cells were observed in intravenous injection of hUCB-MSC, and intravenous or subcutaneous injection of MDP-treated hUCB-MSC. The greatest rate of alleviation of lesions was observed in subcutaneous injection of MDP-treated hUCB-MSC, intravenous injection of MDP-treated hUCB-MSC, and intravenous injection of hUCB-MSC in that order.

11-6: Investigation of the Therapeutic Effect of MDP-Treated hUCB-MSC by Histopathological Examination of Skin Tissue

In order to investigate the therapeutic effect of MDP-treated hUCB-MSCs on atopic dermatitis, an autopsy was conducted after 24 hours of the 4th injection of hUCB-MSCs, and the skin tissues were collected and fixed with a 10% neutral formalin solution. Then, the tissue slices were processed, paraffin-embedded, and cut into 3-4 μm sections. Then, Toluidine blue staining was performed to examine mast cell degranulation, which is one of the major symptoms of atopic dermatitis.

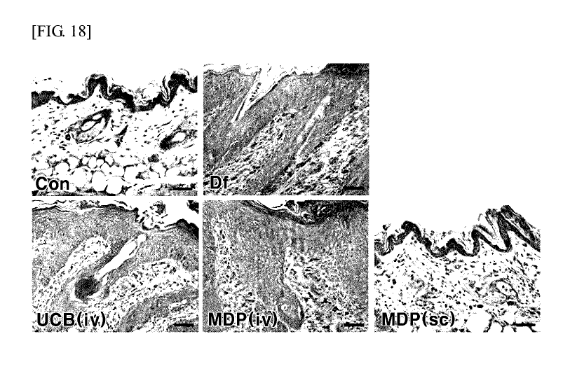

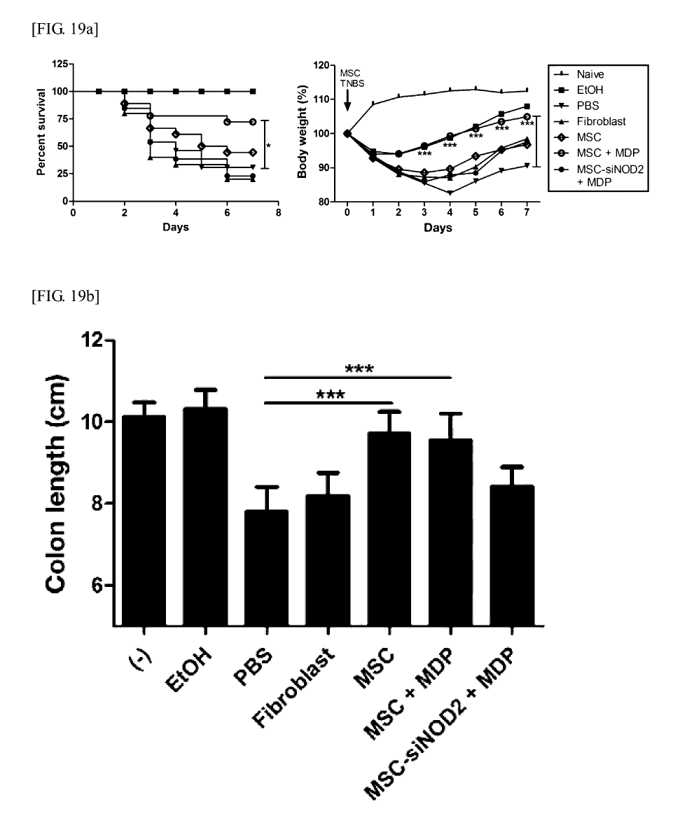

As shown in FIG. 18, a large number of degranulated mast cells were observed in the atopic dermatitis-induced group, and reduction in mast cell degranulation was observed in all of the other groups.

These results suggest that the MDP-treated stem cells of the present invention or the culture thereof can be practically used for the treatment of immune disorders in animal models such as atopic dermatitis.

### EXAMPLE 12

### NOD2 Activation Enhanced the Therapeutic Effect of hUCB-MSCs

12-1: Isolation and Culture of hUCB-MSCs

The UCB samples were obtained from the umbilical vein immediately after delivery, with the informed consent of the mother and approved by the Boramae Hospital Institutional Review Board and the Seoul National University Institutional Review Board (0603/001-002-10C4). The UCB samples were mixed with Hetasep solution (StemCell Technologies, Vancouver, Canada) at a ratio of 5:1, and then incubated at room temperature to deplete erythrocyte counts. The supernatant was collected carefully and mononuclear cells were obtained using Ficoll (GE healthcare life sciences, Pittsburgh, Pa.) density-gradient centrifugation at 2500 rpm for 20 minutes. The cells were washed twice in PBS. Cells were seeded at a density of 2×10⁵ to 2×10⁶ cells/cm2 on plates in growth media that consisted of D-media (formula 78-5470EF; Gibco BRL, Grand Island, N.Y.) containing EGM-2 SingleQuot and 10% fetal bovine serum (Gibco BRL). After 3 days, non-adherent cells were removed. The adherent cells formed colonies and grew rapidly, showing spindle-shaped morphology.

12-2: Mice

C57BL/6J mice (male; age, 8-10 wk) were obtained from Jackson Laboratory (Bar Harbor, Me.) and BALB/c mice (male, 8-10 wk old) from SLC (Hamamatsu, Japan). Mice were group-housed under specific pathogenic-free conditions in the animal facility of Seoul National University.

All experiments were approved by and followed the regulations of the Institute of Laboratory Animals Resources (SNU-100125-8, SNU-111223-1, and SNU-130130-2 Seoul National University).

12-3: RNA Interference

Transfection of siRNA into the cells was conducted when they had reached 60% confluence. The siRNAs of NOD2 (siNOD2, J-011388-07) and non-targeting control (siControl 1 (siCTL), D-001810-01) were purchased from Dharmacon (Chicago, Ill.). Experiments were conducted using DharmaFECT1 (Dharmacon) as a transfection agent and siRNA at a concentration of 100 nmol/L. After 48 hours, the medium was changed and the cells were treated with or without each agonist.

12-4: Statistical Analysis

Mean values among different groups were expressed as mean±SD. All of the statistical comparisons were made by one-way analysis of variance followed by a Bonferroni post hoc test for multigroup comparisons using GraphPad Prism software (version 5.01; GraphPad Software, San Diego, Calif.). Statistical significance designated as asterisks is indicated in the Figure legends.

12-5: NOD2 Activation Enhanced the Therapeutic Effect of hUCB-MSCs Against TNBS-Induced Colitis in Mice

In order to investigate the activation of NOD2 is required for the ability of hUCB-MSCs to reduce the severity of colitis in mice, the effect of hUCB-MSCs on TNBS-induced colitic mice was examined.

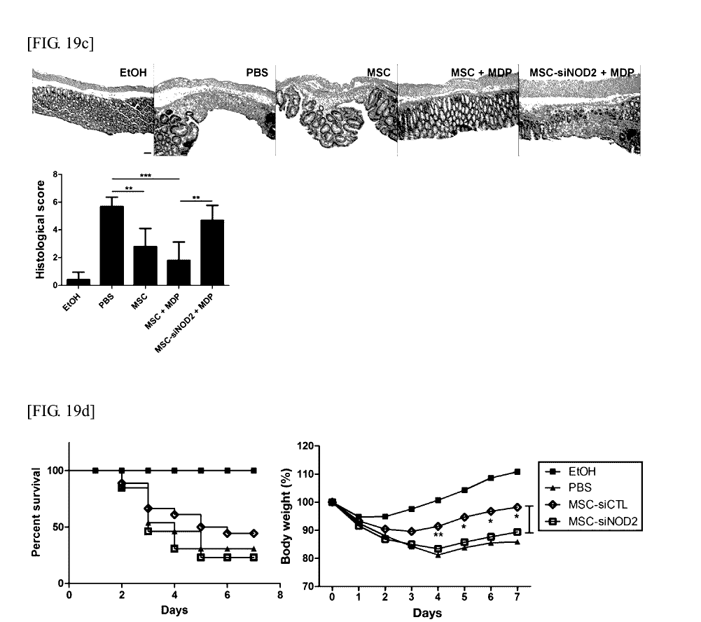

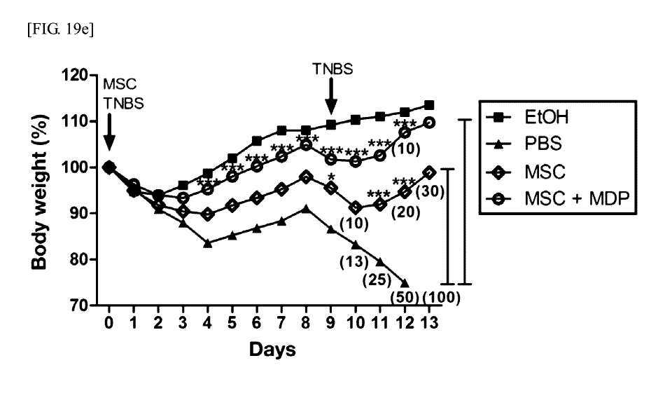

Infusion of hUCB-MSCs increased the survival rate and decreased the loss of body weight (FIG. 19a). MDP-MSCs further improved survival and ameliorated the loss of body weight (FIG. 19a). In addition, shortening of the colon length was significantly prevented by the administration of either hUCB-MSCs or MDP-MSCs (FIG. 19b). Histologic damage also was ameliorated by the injection of hUCB-MSCs, and was ameliorated further by MDP-MSCs (FIG. 19c).

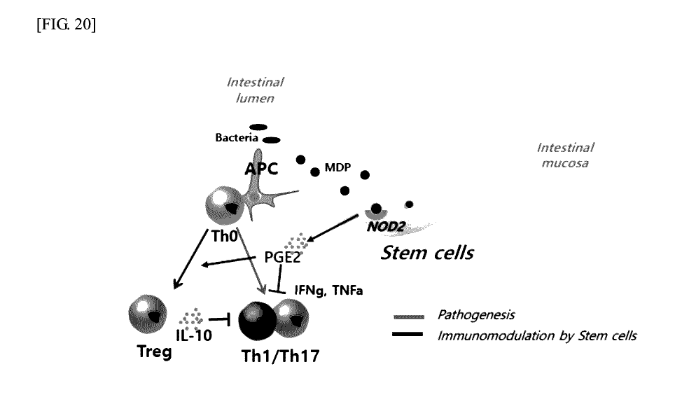

As shown in FIG. 20, NOD2 recognizes bacterial muramyl dipeptide (MDP) in the body of an immune diseases or an inflammatory diseases patient. MDP is the major component of peptidoglycan (PGN) and it is present in both Gram+ and Gram− bacteria. The binding of MDP to NOD2 results in PGE2 and IL-10 production, thereby administration of stem cells expressing NOD2 has a therapeutic effect on immune diseases or inflammatory diseases.

These therapeutic effects of MDP-MSCs were abolished when NOD2 was down-regulated (FIG. 19a-c). Moreover, NOD2 deficiency in hUCB-MSCs resulted in a loss of their protective activity against TNBS-induced colitis because siRNA-induced NOD2 down-regulation in hUCB-MSCs decreased the survival rate and increased the body weight loss in TNBS-treated mice (FIG. 19d). To investigate the generation of immune tolerance in colitic mice by hUCB-MSCs, we assayed whether colitic mice treated initially with hUCB-MSCs or MDP-MSCs could resist a second dose of TNBS without additional treatment with cells. Interestingly, although all mice rapidly died after exposure to the second dose of TNBS, the initial inoculation of hUCB-MSCs protected mice from disease recurrence (FIG. 19e). Infusions with MDP-MSCs led to the amelioration of body weight loss and mortality to a greater extent (FIG. 19e). These results support the model that NOD2 stimulation plays a crucial role in enhancing the immunomodulatory ability of hUCBMSCs, more importantly, these cells cannot maintain their immunomodulatory ability without NOD2.

According to FIG. 19a to 19e, NOD2 is crucial for the protective ability of hUCB-MSCs against TNBS-induced colitis.

As shown in FIG. 19a to 19c, gross and histologic observations in TNBS-induced colitic mice were performed. Numbers of mice were as follows: naive mice, 7; EtOH mice, 8; PBS mice, 13; fibroblast mice, 15; MSC mice, 18; MSC+MDP mice (hUCB-MSCs were cultured in the presence of MDP (10 mg/mL) for 24 hours), 18; and MSC-siNOD2+MDP mice, 13.

NOD2-deficient hUCB-MSCs without MDP stimulation were injected intraperitoneally into colitic mice. Percentage of survival rate and body weight loss were measured (FIG. 19d). Numbers of mice were as follows: EtOH mice, 8; PBS mice, 13; MSC-siCTL mice (CTL means a control), 18; and MSC-siNOD2 mice, 13.

Nine days after colitis induction and hUCB-MSC administration, a second dose of TNBS was inoculated and survival rate was analyzed (FIG. 19e). Numbers of mice were as follows: EtOH mice, 9; PBS mice, 8; MSC mice, 10; and MSC+MDP mice, 10. Numbers in parentheses represent the percentage of mice that were dead. *P<0.05, **P<0.01, ***P<0.001. Results are shown as mean±SD.

Unless otherwise specified, all technical and scientific terms used herein have the same meaning as commonly understood by those of ordinary skill in the art, to which this invention belongs. The nomenclature used herein are also well known and commonly used in the art.

### Effect of the Invention

The present invention provides a pharmaceutical composition that can be used for the prevention or treatment of immune disorders and inflammatory diseases. Furthermore, the present invention provides a method for preparing PGE2 and TGF-β1, which is able to produce PGE2 and TGF-β1 at high yield in a cost-effect way without performing chemical processing. The pharmaceutical composition of the present invention is an inexpensive cellular therapeutic agent having no side-effects, which can be used as an alternative to the previously known immunosuppressive drugs and anti-inflammatory drugs having side-effects. Therefore, it can be used for the prevention or treatment of immune disorders such as autoimmune diseases including Crohn's disease, rheumatoid arthritis, and atopic dermatitis, and inflammatory diseases.

What is claimed is:

1. A method for treatment of immune diseases or inflammatory diseases, comprising steps of
(a) preparing isolated stem cells in which the expression of NOD2 is determined; and
(b) administering the stem cells of step (a) or a culture thereof to the subject.

2. The method according to claim 1, wherein the immune disorders diseases or inflammatory diseases are autoimmune diseases, transplant rejection, arthritis, graft-versus-host-disease, bacterial infection, sepsis or inflammation.

3. The method according to claim 1, wherein the autoimmune diseases are selected from the group consisting of Crohn's disease, erythema, atopic dermatitis, rheumatoid arthritis, Hashimoto's thyroiditis, pernicious anemia, Addison's disease, type 1 diabetes, lupus, chronic fatigue syndrome, fibromyalgia, hypothyroidism and hyperthyroidism, scleroderma, Behcet's disease, inflammatory bowel disease, multiple sclerosis, myasthenia gravis, Meniere's syndrome, Guilian-Barre syndrome, Sjogren's syndrome, vitiligo, endometriosis, psoriasis, vitiligo, systemic scleroderma, asthma, and ulcerative colitis.

4. The method according to claim 1, wherein the stem cells are human adult stem cells, human pluripotent stem cells, induced pluripotent stem cells, animal embryonic stem cells or animal adult stem cells.

5. The method according to claim 4, wherein the adult stem cells are mesenchymal stem cells, human tissue-derived mesenchymal stromal cell, human tissue-derived mesenchymal stem cells, pluripotent stem cells or amniotic epithelial cells.

6. The method according to claim 4, wherein the adult stem cells are mesenchymal stem cells selected from the group consisting of umbilical cord-derived mesenchymal stem cells, umbilical cord blood-derived mesenchymal stem cells, bone marrow-derived mesenchymal stem cells, adipose tissue-derived mesenchymal stem cells, muscle-derived mesenchymal stem cells, nerve-derived mesenchymal stem cells, skin-derived mesenchymal stem cells, amnion-derived mesenchymal stem cells, and placenta-derived mesenchymal stem cells.

7. The method according to claim 1, wherein the step is performed by intra-abdominal, intraarterial injection, intravenous injection, direct injection into the lesion, or injection into the synovial cavity.

8. A pharmaceutical composition for the prevention or treatment of immune diseases or inflammatory diseases, comprising isolated mesenchymal stem cells expressing Nucleotide-binding Oligomerization Domain protein 2 (NOD2) or a culture thereof.

9. The pharmaceutical composition according to claim 8, wherein the expression of NOD2 is determined in the stem cells.

10. The pharmaceutical composition according to claim 8, further comprising a NOD2 agonist.

11. The pharmaceutical composition according to claim 10, wherein the NOD2 agonist is muramyl dipeptide (MDP).

12. A graft that is prepared by determining expression of Nucleotide-binding Oligomerization Domain protein 2 (NOD2) in isolated mesenchymal stem cells, and culturing the stem cells expressing NOD2 on the graft support.

13. The graft according to claim 12, further comprising a NOD2 agonist.

14. The graft according to claim 13, wherein the NOD2 agonist is muramyl dipeptide (MDP).

15. A method for preparing a graft, comprising the steps of
(a) determining expression of Nucleotide-binding Oligomerization Domain protein 2 (NOD2) in isolated mesenchymal stem cells; and
(b) culturing the stem cells of step (a) in the graft.

16. The method according to claim 15, wherein the stem cells of step (b) is cultured with a NOD2 agonist.

17. The method according to claim 16, wherein the NOD2 agonist is muramyl dipeptide (MDP).

18. The method according to claim 16, further comprising the step of removing stem cells after the culturing step.
